(19) United States
(12) Patent Application Publication
(10) Pub. No.: US 2026/0049055 A1
(43) Pub. Date: Feb. 19, 2026

(54) SOLID FORMS OF MESEMBRINE AND THERAPEUTIC USES THEREOF

(71) Applicant: Sensorium Therapeutics, Inc., Boston, MA (US)

(72) Inventor: Zhongyi Lu, Suzhou (CN)

(21) Appl. No.: 18/807,237

(22) Filed: Aug. 16, 2024

Related U.S. Application Data
(63) Continuation of application No. PCT/US23/27746, filed on Jul. 14, 2023.

(30) Foreign Application Priority Data
Jul. 15, 2022 (WO) PCT/CN22/105859

(51) Int. Cl. C07D 209/12; A61K 31/404

(52) U.S. Cl. CPC C07D 209/12; A61K 31/404

(57) ABSTRACT

Solid forms of mesembrine are provided, along with related methods of manufacture. The solid form compositions are useful, for example, in the preparation of pharmaceutical compositions.

### PRIORITY CLAIM

This application claims priority to PCT Application No. PCT/CN22/105859, filed Jul. 15, 2022, the contents of which are incorporated herein by reference in their entirety.

### TECHNICAL FIELD

This disclosure relates to solid forms of mesembrine, and related therapeutic methods of inhibiting the sodium-dependent serotonin transporter (SERT).

### BACKGROUND

Plants of the genus Sceletium contain indole alkaloids having biological activity useful in treating mental health conditions such as mild to moderate depression. Natural extracts of Sceletium tortuosum, an indigenous herb of South Africa also referred to as “kougoed”, “channa” or “kanna,” can contain the pharmacologically active alkaloids. Mesembrine and mesembrenol are pharmacologically active alkaloids present in Sceletium tortuosum extracts used for treatment of anxiety, stress and mental health conditions.

Natural products obtained from plants of the genus Sceletium contain varying amounts of (−) mesembrine and (+)/(−) mesembrenone. The structure of mesembrine, also known as 3a-(3,4-dimethoxyphenyl)-octahydro-1-methyl-6H-indol-6-one, has been reported by Popelak et al., Naturwiss.47,156 (1960), and the configuration by P W Jeffs et al., J. Am. Chem. Soc. 91, 3831 (1969). Naturally occurring (−) mesembrine (Compound 1) from Sceletium tortuosum has been reported as having serotonin (5-HT) uptake inhibitory activity useful in treating mental health conditions such as mild to moderate depression. Naturally occurring (+)/(−) mesembrenone from Sceletium tortuosum is reported as a potent selective serotonin reuptake inhibitor (Ki=27 nM).

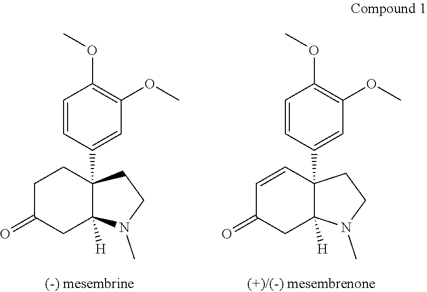 [SMILES: COc1ccc([C@@]23CCC(=O)C[C@@H]2N(C)CC3)cc1OC ; COc1ccc([C@@]23C=CC(=O)C[C@@H]2N(C)CC3)cc1OC]

Polymorphs, solvates and salts of various drugs have been described in the literature as imparting novel properties to the drugs. Organic small drug molecules have a tendency to self-assemble into various polymorphic forms depending on the environment that drives the self-assembly. Heat and solvent mediated effects can also lead to changes that transform one polymorphic form into another.

Identifying which polymorphic form is the most stable under each condition of interest and the processes that lead to changes in the polymorphic form is crucial to the design of the drug manufacturing process in order to ensure that the final product is in its preferred polymorphic form. Different polymorphic forms of an active pharmaceutical ingredient (API) can lead to changes in the drug's solubility, dissolution rate, pharmacokinetics, and ultimately its bioavailability and efficacy in patients.

### SUMMARY OF THE INVENTION

Described are solid forms of mesembrine (e.g., (−) mesembrine (Compound 1)). In some embodiments, solid forms of mesembrine comprise the product of the processes disclosed herein.

In some embodiments, a pharmaceutical composition comprises a solid form described herein; and a pharmaceutically acceptable excipient.

In some embodiments, a pharmaceutical composition is formed by a process comprising dissolving a solid form described herein.

In some embodiments, a method of treating a mental health disorder, comprises administering to a mammal in need thereof an effective amount of a solid form described herein or a pharmaceutical composition described herein. In some embodiments, the mental health disorder is anxiety, stress, or depression. In some embodiments, the mammal is a human.

### BRIEF DESCRIPTION OF THE DRAWINGS

FIG. 1A is an X-Ray powder diffraction (XRPD) pattern obtained from sample of solid Tartrate Form B of Compound 1.

FIG. 1B is a TGA thermogram of material analyzed from room temperature to 350° C. at 10° C. per minute obtained from sample of solid Tartrate Form B of Compound 1.

FIG. 1C is a DSC thermogram of material analyzed from room temperature to 230° C. at 10° C. per minute obtained from sample of solid Tartrate Form B of Compound 1.

FIG. 2A is an X-Ray powder diffraction (XRPD) pattern obtained from sample of solid Fumarate Form A of Compound 1.

FIG. 2B is a TGA thermogram of material analyzed from room temperature to 350° C. at 10° C. per minute obtained from sample of solid Fumarate Form A of Compound 1.

FIG. 2C is a DSC thermogram of material analyzed from room temperature to 230° C. at 10° C. per minute obtained from sample of solid Fumarate Form A of Compound 1.

FIG. 3A is an X-Ray powder diffraction (XRPD) pattern obtained from sample of solid Malate Form A of Compound 1.

FIG. 3B is a TGA thermogram of material analyzed from room temperature to 350° C. at 10° C. per minute obtained from sample of solid Malate Form A of Compound 1.

FIG. 3C is a DSC thermogram of material analyzed from room temperature to 230° C. at 10° C. per minute obtained from sample of solid Malate Form A of Compound 1.

FIG. 4A is an X-Ray powder diffraction (XRPD) pattern obtained from sample of solid Malate Form C of Compound 1.

FIG. 4B is a TGA thermogram of material analyzed from room temperature to 350° C. at 10° C. per minute obtained from sample of solid Malate Form C of Compound 1.

FIG. 4C is a DSC thermogram of material analyzed from room temperature to 230° C. at 10° C. per minute obtained from sample of solid Malate Form C of Compound 1.

FIG. 5A is an X-Ray powder diffraction (XRPD) pattern obtained from sample of solid Succinate Form A of Compound 1.

FIG. 5B is a TGA thermogram of material analyzed from room temperature to 350° C. at 10° C. per minute obtained from sample of solid Succinate Form A of Compound 1.

FIG. 5C is a DSC thermogram of material analyzed from room temperature to 230° C. at 10° C. per minute obtained from sample of solid Succinate Form A of Compound 1.

FIG. 6A is an X-Ray powder diffraction (XRPD) pattern obtained from sample of solid Succinate Form B of Compound 1.

FIG. 6B is a TGA thermogram of material analyzed from room temperature to 350° C. at 10° C. per minute obtained from sample of solid Succinate Form B of Compound 1.

FIG. 6C is a DSC thermogram of material analyzed from room temperature to 230° C. at 10° C. per minute obtained from sample of solid Succinate Form B of Compound 1.

FIG. 7A is an X-Ray powder diffraction (XRPD) pattern obtained from sample of solid Naphthalene Disulfonate Form A of Compound 1.

FIG. 7B is a TGA thermogram of material analyzed from room temperature to 350° C. at 10° C. per minute obtained from sample of solid Naphthalene Disulfonate Form A of Compound 1.

FIG. 7C is a DSC thermogram of material analyzed from room temperature to 230° C. at 10° C. per minute obtained from sample of solid Naphthalene Disulfonate Form A of Compound 1.

FIG. 8A is an X-Ray powder diffraction (XRPD) pattern obtained from sample of solid HBr Form A of Compound 1.

FIG. 8B is a TGA thermogram of material analyzed from room temperature to 350° C. at 10° C. per minute obtained from sample of solid HBr Form A of Compound 1.

FIG. 8C is a DSC thermogram of material analyzed from room temperature to 230° C. at 10° C. per minute obtained from sample of solid HBr Form A of Compound 1.

### DETAILED DESCRIPTION OF THE INVENTION

Applicants have discovered solid forms of mesembrine (e.g., (−) mesembrine (Compound 1)). Although (−) mesembrine is bioactive with certain desirable pharmacologic effects, certain other properties are less than ideal for use as a therapeutic. Solid forms of mesembrine (e.g., crystalline salts of mesembrine) are described herein.

Mesembrine can occur in solid forms as an amorphous solid form or in a crystalline solid form or in mixtures of solid forms. Crystalline solid forms of mesembrine can exist in one or more unique solid forms, which can additionally comprise one or more equivalents of water or solvent (i.e., hydrates or solvates, respectively).

Crystalline form(s) of mesembrine having distinct characteristic XRPD peaks are provided herein. Accordingly, provided herein are crystalline mesembrine solid forms, pharmaceutical compositions thereof, and methods of preparing those crystalline mesembrine solid forms and methods of use thereof.

Compositions comprising the free base of Compound 1 in certain solid forms are provided. Compound 1 can occur in solid forms as an amorphous solid form or in a crystalline solid form or in mixtures of solid forms. Crystalline solid forms of Compound 1 can exist in one or more unique solid forms, which can additionally comprise one or more equivalents of water or solvent (i.e., hydrates or solvates, respectively). Crystalline form(s) of Compound 1 having distinct characteristic XRPD peaks are provided herein. Accordingly, provided herein are crystalline Compound 1 solid forms, pharmaceutical compositions thereof, and methods of preparing those crystalline Compound 1 solid forms and methods of use thereof.

### Salt Forms of Compound 1

Various crystalline salts of Compound 1 were prepared, including (but not limited to) crystalline tartrate, fumarate, malate, succinate, naphthalene disulfonate, and HBr forms. In some embodiments, a composition comprises a solid form of mesembrine (e.g., Compound 1 (−) mesembrine) in combination with one or more compounds selected from the group consisting of tartaric acid, fumaric acid, malic acid, succinic acid, naphthalene disulfonic acid, and hydrobromic acid.

### Tartrate Form B

In some embodiments, solid forms of a tartrate salt of Compound 1 are provided. Tartrate Form B is a solid form of the tartrate salt of Compound 1 that can be identified by a XRPD pattern comprising certain characteristic peaks, and/or by one or more other techniques including DSC, TGA, and/or DVS.

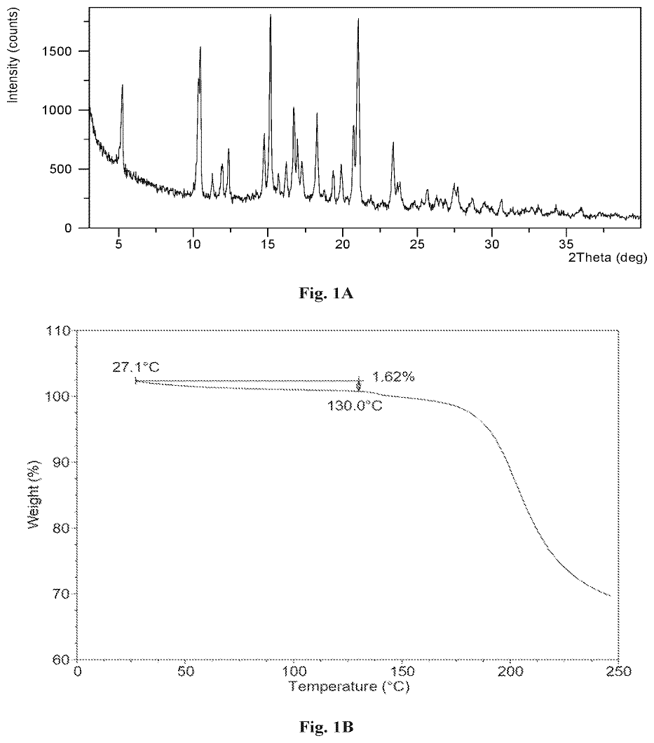

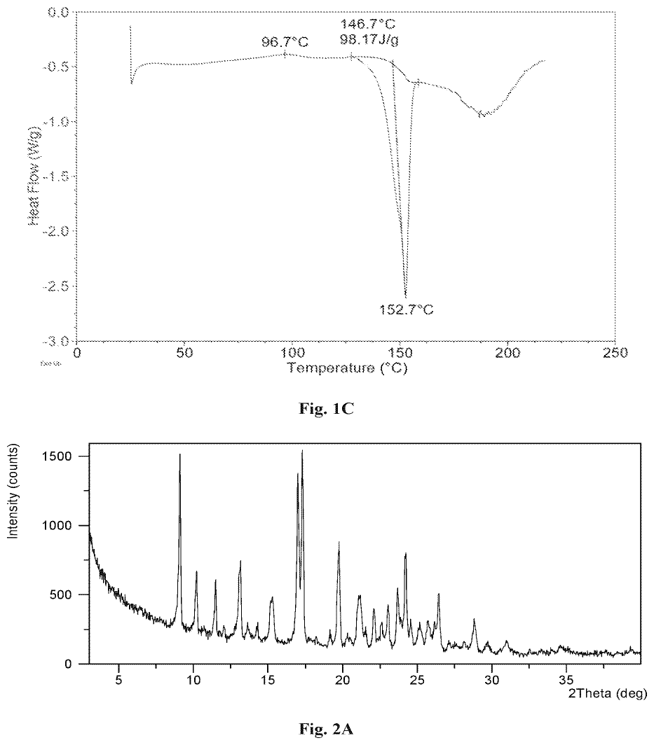

Samples of Tartrate Form B were prepared according to the Example 2 and characterized by XRPD (e.g., a Pattern the same or substantially similar to or not dissimilar to FIG. 1A), TGA (e.g., a thermogram the same or substantially similar to or not dissimilar to FIG. 1B), and DSC (e.g., a thermogram the same or substantially similar to or not dissimilar to FIG. 1C).

In some embodiments, a solid tartrate salt form of mesembrine, such as Tartrate Form B, has an X-ray powder diffraction (XRPD) pattern comprising 2-theta peaks (2 theta±0.2) 10.5°, 15.2°, 16.7°, 18.3°, and 21.0°. In some embodiments, a solid tartrate salt form of mesembrine, such as Tartrate Form B, has an X-ray powder diffraction (XRPD) pattern comprising 2-theta peaks (2 theta±0.2) 5.2°, 10.5°, 11.3°, 12.0°, 12.4°, 14.8°, 15.2°, 15.7°, 16.2°, 16.7°, 17.0°, 17.3°, 18.3°, 19.4°, 19.9°, 20.7°, 21.0°, 23.4°, 23.8°, 25.7°, and 27.4°.

In some embodiments, a solid tartrate salt form of mesembrine, such as Tartrate Form B, has an X-ray powder diffraction (XRPD) pattern comprising 2-theta peaks according to Table 5 having a relative intensity of greater than 10%. In some embodiments, a solid tartrate salt form of mesembrine, such as Tartrate Form B, has an X-ray powder diffraction (XRPD) pattern according to FIG. 1A.

In some embodiments, a solid tartrate salt form of mesembrine, such as Tartrate Form B, is characterized by an X-ray powder diffraction (XRPD) having 2-theta peaks (2 theta±0.2) and corresponding d-spacing (angstroms±0.2):

| 2θ ± 0.2 [°] | d-spacing ± 0.2 [Å] |
|---|---|
| 10.5 | 8.4 |
| 15.2 | 5.8 |
| 16.7 | 5.3 |
| 18.3 | 4.8 |
| 21.0 | 4.2. |

In some embodiments, a solid tartrate salt form of mesembrine, such as Tartrate Form B, is characterized by an X-ray powder diffraction (XRPD) having 2-theta peaks (2 theta±0.2) and corresponding d-spacing (angstroms±0.2):

| 2θ[°] | d-spacing [Å] |
|---|---|
| 5.2 | 16.9 |
| 10.5 | 8.4 |
| 11.3 | 7.8 |
| 12.0 | 7.4 |
| 12.4 | 7.2 |
| 14.8 | 6.0 |
| 15.2 | 5.8 |
| 15.7 | 5.6 |
| 16.2 | 5.5 |
| 16.7 | 5.3 |
| 17.0 | 5.2 |
| 17.3 | 5.1 |
| 18.3 | 4.8 |
| 19.4 | 4.6 |
| 19.9 | 4.5 |
| 20.7 | 4.3 |
| 21.0 | 4.2 |
| 23.4 | 3.8 |
| 23.8 | 3.7 |
| 25.7 | 3.5 |
| 27.4 | 3.2. |

In some embodiments, a solid tartrate salt form of mesembrine, such as Tartrate Form B, is characterized by a TGA thermogram according to FIG. 1B. In some embodiments, a solid tartrate salt form of mesembrine, such as Tartrate Form B, is further characterized by a sample weight loss of up to about 1.6% upon heating up to 130° C. measured by TGA according to the following parameters:

| Parameters | TGA | |
|---|---|---|
| Method | Ramp | |
| Sample pan | Aluminum, open | |
| Temperature | RT-350° | C. |
| Heating rate | 10° | C./min |

| | |
|---|---|
| Purge gas | N₂. |

In some embodiments, a solid tartrate salt form of mesembrine, such as Tartrate Form B, is characterized by a differential scanning calorimetry (DSC) thermogram according to FIG. 1C. In some embodiments, a solid tartrate salt form of mesembrine, such as Tartrate Form B, is further characterized by a DSC having an endotherm at about 150° C. to about 155° C. peak temperature), measured by DSC according to the following parameters:

| | |
|---|---|
| Parameters | DSC |
| Method | Ramp |
| Sample pan | Aluminum, crimped |
| Temperature | 25° C.-230° C. |
| Heating rate | 10° C./min |
| Purge gas | N₂. |

### Fumarate Form A

In some embodiments, solid forms of a fumarate salt of Compound 1 are provided. Fumarate Form A is a solid form of the fumarate salt of Compound 1 that can be identified by a XRPD pattern comprising certain characteristic peaks, and/or by one or more other techniques including DSC, TGA, and/or DVS.

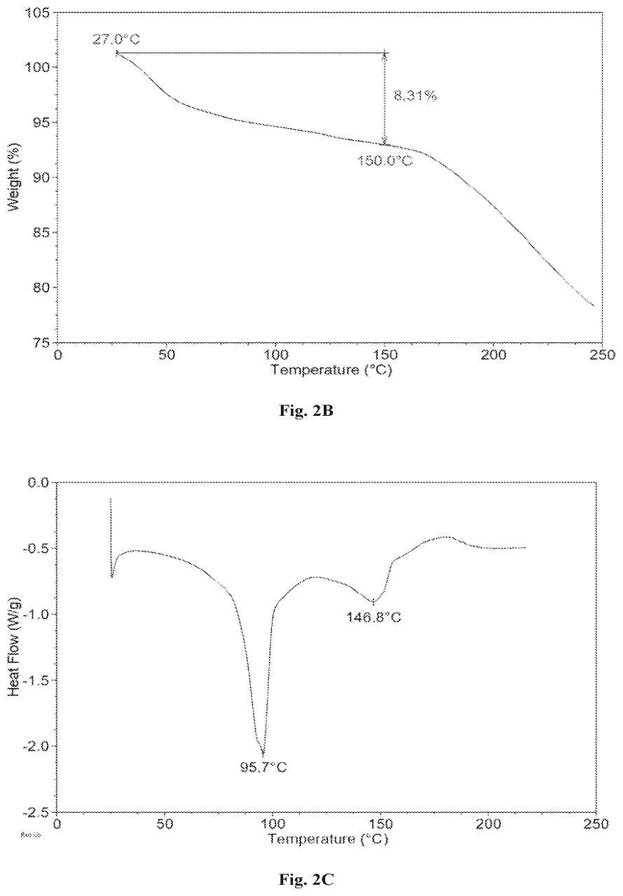

Samples of Fumarate Form A were prepared according to the Example 3 and characterized by XRPD (e.g., a Pattern the same or substantially similar to or not dissimilar to FIG. 2A), TGA (e.g., a thermogram the same or substantially similar to or not dissimilar to FIG. 2B), and DSC (e.g., a thermogram the same or substantially similar to or not dissimilar to FIG. 2C).

In some embodiments, a solid fumarate salt form of mesembrine, such as Fumarate Form A, has an X-ray powder diffraction (XRPD) pattern comprising 2-theta peaks (2 theta±0.2) 9.1°, 17.0°, 17.3°, 23.7°, and 24.2°. In some embodiments, a solid fumarate salt form of mesembrine, such as Fumarate Form A, has an X-ray powder diffraction (XRPD) pattern comprising 2-theta peaks (2 theta±0.2) 9.1°, 10.2°, 11.5°, 13.2°, 15.3°, 17.0°, 17.3°, 19.8°, 21.1°, 22.1°, 22.6°, 23.1°, 23.7°, 24.2°, 24.6°, 25.2°, 25.7°, 26.4°, and 28.8°.

In some embodiments, a solid fumarate salt form of mesembrine, such as Fumarate Form A, has an X-ray powder diffraction (XRPD) pattern comprising 2-theta peaks according to Table 6 having a relative intensity of greater than 10%. In some embodiments, a solid fumarate salt form of mesembrine, such as Fumarate Form A, has an X-ray powder diffraction (XRPD) pattern according to FIG. 2A.

In some embodiments, a solid fumarate salt form of mesembrine, such as Fumarate Form A, is characterized by an X-ray powder diffraction (XRPD) having 2-theta peaks (2 theta±0.2) and corresponding d-spacing (angstroms±0.2):

| | d-spacing ± 0.2 |
|---|---|
| 2θ ± 0.2 [°] | [Å] |
| 9.1 | 9.7 |
| 17 | 5.2 |
| 17.3 | 5.1 |
| 23.7 | 3.8 |
| 24.2 | 3.7. |

In some embodiments, a solid fumarate salt form of mesembrine, such as Fumarate Form A, is characterized by an X-ray powder diffraction (XRPD) having 2-theta peaks (2 theta±0.2) and corresponding d-spacing (angstroms±0.2):

| | d-spacing ± 0.2 |
|---|---|
| 2θ ± 0.2 [°] | [Å] |
| 9.1 | 9.7 |
| 10.2 | 8.6 |
| 11.5 | 7.7 |
| 13.2 | 6.7 |
| 15.3 | 5.8 |
| 17.0 | 5.2 |
| 17.3 | 5.1 |
| 19.8 | 4.5 |
| 21.1 | 4.2 |
| 22.1 | 4.0 |
| 22.6 | 3.9 |
| 23.1 | 3.9 |
| 23.7 | 3.8 |
| 24.2 | 3.7 |
| 24.6 | 3.6 |
| 25.2 | 3.5 |
| 25.7 | 3.5 |
| 26.4 | 3.4 |
| 28.8 | 3.1. |

In some embodiments, a solid fumarate salt form of mesembrine, such as Fumarate Form A, is characterized by a TGA thermogram according to FIG. 2B. In some embodiments, a solid fumarate salt form of mesembrine, such as Fumarate Form A, is further characterized by a sample weight loss of up to about 8.3% upon heating up to 150° C. measured by TGA according to the following parameters:

| Parameters | TGA | |
|---|---|---|
| Method | Ramp | |
| Sample pan | Aluminum, open | |
| Temperature | RT-350° | C. |
| Heating rate | 10° | C./min |

| | |
|---|---|
| Purge gas | N₂. |

In some embodiments, a solid fumarate salt form of mesembrine, such as Fumarate Form A, is characterized by a differential scanning calorimetry (DSC) thermogram according to FIG. 2C. In some embodiments, a solid fumarate salt form of mesembrine, such as Fumarate Form A, is further characterized by a DSC having an endotherm at about 92° C. to about 97° C. (peak temperature) and/or an endotherm at about 145° C. to about 150° C. (peak temperature), measured by DSC according to the following parameters:

| | |
|---|---|
| Parameters | DSC |
| Method | Ramp |
| Sample pan | Aluminum, crimped |
| Temperature | 25° C.-230° C. |
| Heating rate | 10° C./min |
| Purge gas | N₂. |

### Malate Form A

In some embodiments, solid forms of a malate salt of Compound 1 are provided. Malate Form A is a solid form of the malate salt of Compound 1 that can be identified by a XRPD pattern comprising certain characteristic peaks, and/or by one or more other techniques including DSC, TGA, and/or DVS.

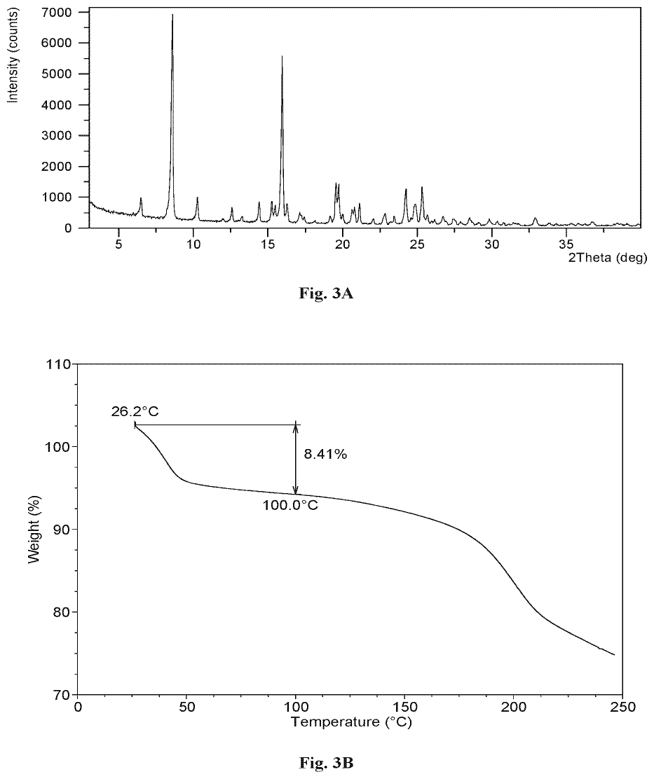

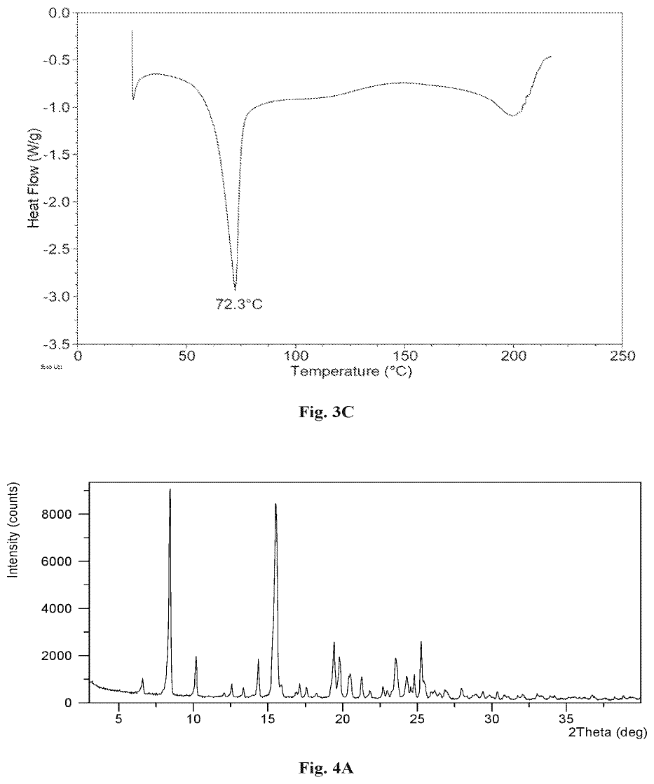

Samples of Malate Form A were prepared according to the Example 4 and characterized by XRPD (e.g., a Pattern the same or substantially similar to or not dissimilar to FIG. 3A), TGA (e.g., a thermogram the same or substantially similar to or not dissimilar to FIG. 3B), and DSC (e.g., a thermogram the same or substantially similar to or not dissimilar to FIG. 3C).

In some embodiments, a solid malate salt form of mesembrine, such as Malate Form A, has an X-ray powder diffraction (XRPD) pattern comprising 2-theta peaks (2 theta±0.2) 9.1°, 17.0°, 17.3°, 23.7°, and 24.2°. In some embodiments, a solid malate salt form of mesembrine, such as Malate Form A, has an X-ray powder diffraction (XRPD) pattern comprising 2-theta peaks (2 theta±0.2) 9.1°, 10.2°, 11.5°, 13.2°, 15.3°, 17.0°, 17.3°, 19.8°, 21.1°, 22.1°, 22.6°, 23.1°, 23.7°, 24.2°, 24.6°, 25.2°, 25.7°, 26.4°, and 28.8°.

In some embodiments, a solid malate salt form of mesembrine, such as Malate Form A, has an X-ray powder diffraction (XRPD) pattern comprising 2-theta peaks according to Table 7 having a relative intensity of greater than 10%. In some embodiments, a solid malate salt form of mesembrine, such as Malate Form A, has an X-ray powder diffraction (XRPD) pattern according to FIG. 3A.

In some embodiments, a solid malate salt form of mesembrine, such as Malate Form A, is characterized by an X-ray powder diffraction (XRPD) having 2-theta peaks (2 theta±0.2) and corresponding d-spacing (angstroms±0.2):

| 2θ ± 0.2 [°] | d-spacing ± 0.2 [Å] |
|---|---|
| 9.1 | 9.7 |
| 17 | 5.2 |
| 17.3 | 5.1 |
| 23.7 | 3.8 |
| 24.2 | 3.7. |

In some embodiments, a solid malate salt form of mesembrine, such as Malate Form A, is characterized by an X-ray powder diffraction (XRPD) having 2-theta peaks (2 theta±0.2) and corresponding d-spacing (angstroms±0.2):

| 2θ ± 0.2 [°] | d-spacing ± 0.2 [Å] |
|---|---|
| 9.1 | 9.7 |
| 10.2 | 8.6 |
| 11.5 | 7.7 |
| 13.2 | 6.7 |
| 15.3 | 5.8 |
| 17.0 | 5.2 |
| 17.3 | 5.1 |
| 19.8 | 4.5 |
| 21.1 | 4.2 |
| 22.1 | 4.0 |
| 22.6 | 3.9 |
| 23.1 | 3.9 |
| 23.7 | 3.8 |
| 24.2 | 3.7 |
| 24.6 | 3.6 |
| 25.2 | 3.5 |
| 25.7 | 3.5 |
| 26.4 | 3.4 |
| 28.8 | 3.1. |

In some embodiments, a solid malate salt form of mesembrine, such as Malate Form A, is characterized by a TGA thermogram according to FIG. 3B. In some embodiments, a solid malate salt form of mesembrine, such as Malate Form A, is further characterized by a sample weight loss of up to about 8.3% upon heating up to 150° C. measured by TGA according to the following parameters:

| Parameters | TGA | |
|---|---|---|
| Method | Ramp | |
| Sample pan | Aluminum, open | |
| Temperature | RT-350° | C. |
| Heating rate | 10° | C./min |

| | |
|---|---|
| Purge gas | N₂. |

In some embodiments, a solid malate salt form of mesembrine, such as Malate Form A, is characterized by a differential scanning calorimetry (DSC) thermogram according to FIG. 3C. In some embodiments, a solid malate salt form of mesembrine, such as Malate Form A, is further characterized by a DSC having an endotherm at about 92° C. to about 97° C. (peak temperature) and/or an endotherm at about 145° C. to about 150° C. (peak temperature), measured by DSC according to the following parameters:

| | |
|---|---|
| Parameters | DSC |
| Method | Ramp |
| Sample pan | Aluminum, crimped |
| Temperature | 25° C.-230° C. |
| Heating rate | 10° C./min |
| Purge gas | N₂. |

### Malate Form C

In some embodiments, solid forms of a malate salt of Compound 1 are provided. Malate Form C is a solid form of the malate salt of Compound 1 that can be identified by a XRPD pattern comprising certain characteristic peaks, and/or by one or more other techniques including DSC, TGA, and/or DVS.

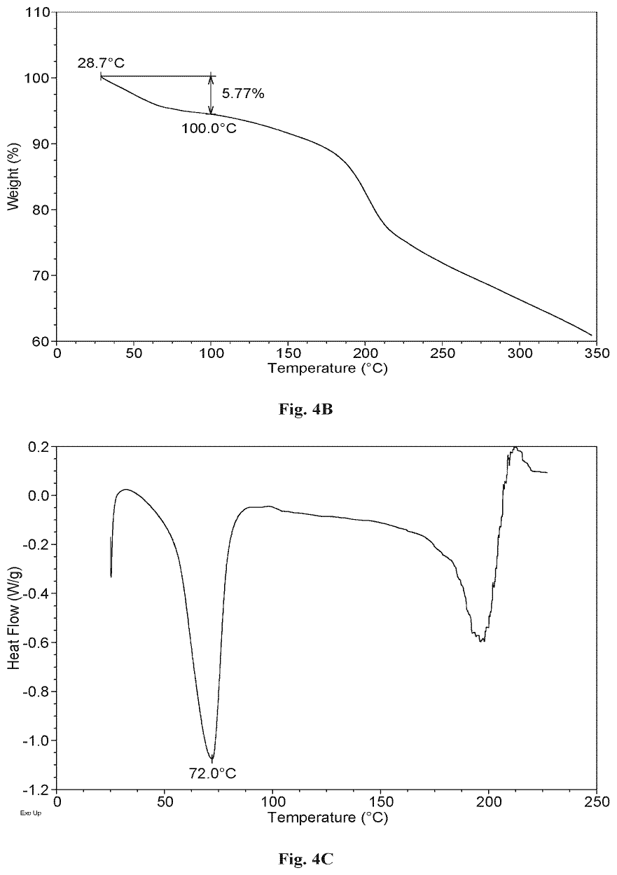

Samples of Malate Form C were prepared according to the Example 5 and characterized by XRPD (e.g., a Pattern the same or substantially similar to or not dissimilar to FIG. 4A), TGA (e.g., a thermogram the same or substantially similar to or not dissimilar to FIG. 4B), and DSC (e.g., a thermogram the same or substantially similar to or not dissimilar to FIG. 4C).

In some embodiments, a solid malate salt form of mesembrine, such as Malate Form C, has an X-ray powder diffraction (XRPD) pattern comprising 2-theta peaks (2 theta±0.2) 8.4°, 15.3°, 15.6°, 19.4°, and 25.2°. In some embodiments, a solid malate salt form of mesembrine, such as Malate Form C, has an X-ray powder diffraction (XRPD) pattern comprising 2-theta peaks (2 theta±0.2) 8.4°, 10.2°, 14.4°, 15.3°, 15.6°, 19.4°, 19.8°, 20.5°, 21.3°, 23.5°, 24.3°, 24.8°, and 25.2°.

In some embodiments, a solid malate salt form of mesembrine, such as Malate Form C, has an X-ray powder diffraction (XRPD) pattern comprising 2-theta peaks according to Table 8 having a relative intensity of greater than 10%. In some embodiments, a solid malate salt form of mesembrine, such as Malate Form C, has an X-ray powder diffraction (XRPD) pattern according to FIG. 4A.

In some embodiments, a solid malate salt form of mesembrine, such as Malate Form C, is characterized by an X-ray powder diffraction (XRPD) having 2-theta peaks (2 theta±0.2) and corresponding d-spacing (angstroms±0.2):

| | d-spacing ± 0.2 |
|---|---|
| 2θ ± 0.2 [°] | [Å] |
| 8.4 | 10.5 |
| 15.3 | 5.8 |
| 15.6 | 5.7 |
| 19.4 | 4.6 |
| 25.2 | 3.5. |

In some embodiments, a solid malate salt form of mesembrine, such as Malate Form C, is characterized by an X-ray powder diffraction (XRPD) having 2-theta peaks (2 theta±0.2) and corresponding d-spacing (angstroms±0.2):

| 2θ[°] | d-spacing [Å] |
|---|---|
| 8.4 | 10.5 |
| 10.2 | 8.7 |
| 14.4 | 6.2 |
| 15.3 | 5.8 |
| 15.6 | 5.7 |
| 19.4 | 4.6 |
| 19.8 | 4.5 |
| 20.5 | 4.3 |
| 21.3 | 4.2 |
| 23.5 | 3.8 |
| 24.3 | 3.7 |
| 24.8 | 3.6 |
| 25.2 | 3.5. |

In some embodiments, a solid malate salt form of mesembrine, such as Malate Form C, is characterized by a TGA thermogram according to FIG. 4B. In some embodiments, a solid malate salt form of mesembrine, such as Malate Form C, is further characterized by a sample weight loss of up to about 8.4% upon heating up to 100° C. measured by TGA according to the following parameters:

| | |
|---|---|
| Parameters | TGA |
| Method | Ramp |
| Sample pan | Aluminum, open |
| Temperature | RT - 350 ° C. |
| Heating rate | 10 °C/min |
| Purge gas | N2. |

In some embodiments, a solid malate salt form of mesembrine, such as Malate Form C, is characterized by a differential scanning calorimetry (DSC) thermogram according to FIG. 4C. In some embodiments, a solid malate salt form of mesembrine, such as Malate Form C, is further characterized by a DSC having an endotherm at about 70° C. to about 75° C. (peak temperature), measured by DSC according to the following parameters:

| | |
|---|---|
| Parameters | DSC |
| Method | Ramp |
| Sample pan | Aluminum, crimped |
| Temperature | 25 ° C. - 230 ° C. |
| Heating rate | 10 ° C./min |
| Purge gas | N2. |

### Succinate Form A

In some embodiments, solid forms of a succinate salt of Compound 1 are provided. Succinate Form A is a solid form of the succinate salt of Compound 1 that can be identified by a XRPD pattern comprising certain characteristic peaks, and/or by one or more other techniques including DSC, TGA, and/or DVS.

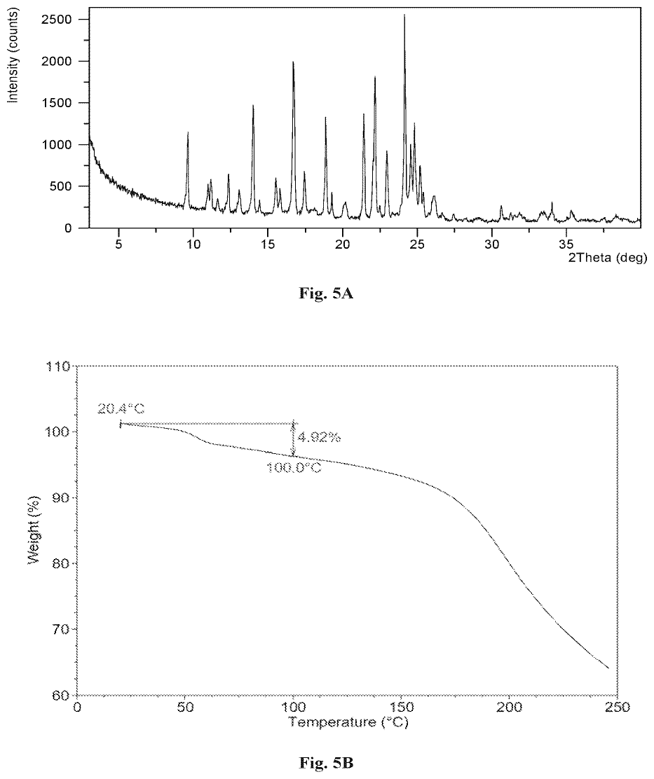

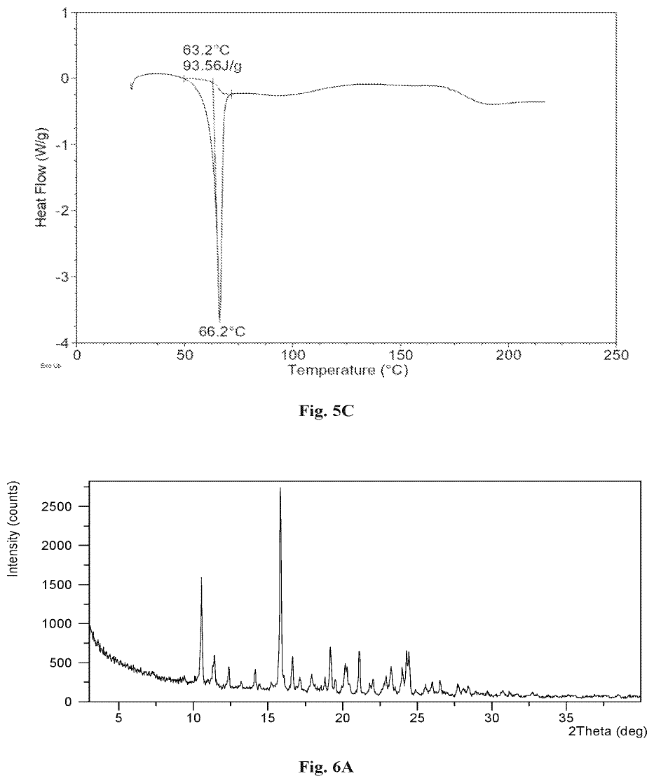

Samples of Succinate Form A were prepared according to the Example 6 and characterized by XRPD (e.g., a Pattern the same or substantially similar to or not dissimilar to FIG. 5A), TGA (e.g., a thermogram the same or substantially similar to or not dissimilar to FIG. 5B), and DSC (e.g., a thermogram the same or substantially similar to or not dissimilar to FIG. 5C).

In some embodiments, a solid succinate salt form of mesembrine, such as Succinate Form A, has an X-ray powder diffraction (XRPD) pattern comprising 2-theta peaks (2 theta±0.2) 14.0°, 16.7°, 21.4°, 22.2°, and 24.2°. In some embodiments, a solid succinate salt form of mesembrine, such as Succinate Form A, has an X-ray powder diffraction (XRPD) pattern comprising 2-theta peaks (2 theta±0.2) 9.6°, 11.0°, 11.3°, 12.4°, 13.1°, 14.0°, 15.5°, 15.8°, 16.7°, 17.4°, 18.9°, 19.3°, 21.4°, 22.2°, 23.0°, 24.2°, 24.6°, 24.8°, 25.2°, 25.4°, and 26.1°.

In some embodiments, a solid succinate salt form of mesembrine, such as Succinate Form A, has an X-ray powder diffraction (XRPD) pattern comprising 2-theta peaks according to Table 9 having a relative intensity of greater than 10%. In some embodiments, a solid succinate salt form of mesembrine, such as Succinate Form A, has an X-ray powder diffraction (XRPD) pattern according to FIG. 5A.

In some embodiments, a solid succinate salt form of mesembrine, such as Succinate Form A, is characterized by an X-ray powder diffraction (XRPD) having 2-theta peaks (2 theta±0.2) and corresponding d-spacing (angstroms±0.2):

| | |
|---|---|
| 20+0.2 [°] | d-spacing +0.2 |
| | [Å] |
| 14.0 | 16.5 |
| 16.7 | 5.3 |
| 21.4 | 4.2 |
| 22.2 | 4.0 |
| 24.2 | 3.7. |

In some embodiments, a solid succinate salt form of mesembrine, such as Succinate Form A, is characterized by an X-ray powder diffraction (XRPD) having 2-theta peaks (2 theta±0.2) and corresponding d-spacing (angstroms±0.2):

| | |
|---|---|
| 20[°] | d-spacing [A] |
| 9.6 | 9.2 |
| 11.0 | 8.0 |
| 11.3 | 7.9 |
| 12.4 | 7.2 |
| 13.1 | 6.8 |
| 14.0 | 6.3 |
| 15.5 | 5.7 |
| 15.8 | 5.6 |
| 16.7 | 5.3 |
| 17.4 | 5.1 |
| 18.9 | 4.7 |
| 19.3 | 4.6 |
| 21.4 | 4.2 |
| 22.2 | 4.0 |
| 23.0 | 3.9 |
| 24.2 | 3.7 |
| 24.6 | 3.6 |
| 24.8 | 3.6 |
| 25.2 | 3.5 |
| 25.4 | 3.5 |
| 26.1 | 3.4. |

In some embodiments, a solid succinate salt form of mesembrine, such as Succinate Form A, is characterized by a TGA thermogram according to FIG. 5B. In some embodiments, a solid succinate salt form of mesembrine, such as Succinate Form A, is further characterized by a sample weight loss of up to about 4.9% upon heating up to 100° C. measured by TGA according to the following parameters:

| | |
|---|---|
| Parameters | TGA |
| Method | Ramp |
| Sample pan | Aluminum, open |
| Temperature | RT - 350 ° C. |
| Heating rate | 10 ° C./min |
| Purge gas | N2. |

In some embodiments, a solid succinate salt form of mesembrine, such as Succinate Form A, is characterized by a differential scanning calorimetry (DSC) thermogram according to FIG. 5C. In some embodiments, a solid succinate salt form of mesembrine, such as Succinate Form A, is further characterized by a DSC having an endotherm at about 64° C. to about 69° C. (peak temperature), measured by DSC according to the following parameters:

| | |
|---|---|
| Parameters | DSC |
| Method | Ramp |
| Sample pan | Aluminum, crimped |
| Temperature | 25 ° C. - 230 ° C. |
| Heating rate | 10 ° C./min |
| Purge gas | N2. |

### Succinate Form B

In some embodiments, solid forms of a succinate salt of Compound 1 are provided. Succinate Form B is a solid form of the succinate salt of Compound 1 that can be identified by a XRPD pattern comprising certain characteristic peaks, and/or by one or more other techniques including DSC, TGA, and/or DVS.

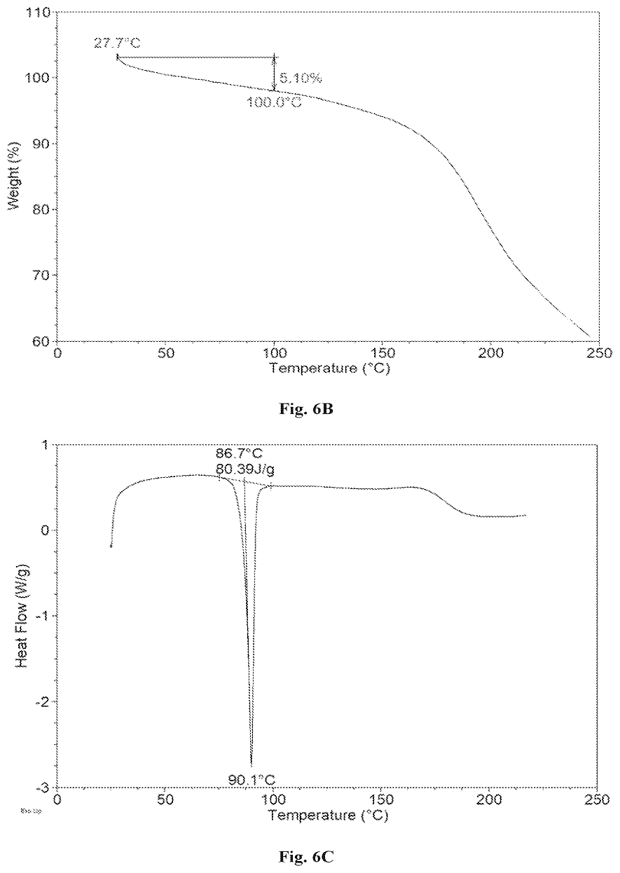

Samples of Succinate Form B were prepared according to the Example 7 and characterized by XRPD (e.g., a Pattern the same or substantially similar to or not dissimilar to FIG. 6A), TGA (e.g., a thermogram the same or substantially similar to or not dissimilar to FIG. 6B), and DSC (e.g., a thermogram the same or substantially similar to or not dissimilar to FIG. 6C).

In some embodiments, a solid succinate salt form of mesembrine, such as Succinate Form B, has an X-ray powder diffraction (XRPD) pattern comprising 2-theta peaks (2 theta±0.2) 10.6°, 15.8°, 19.2°, 21.1°, and 24.3°. In some embodiments, a solid succinate salt form of mesembrine, such as Succinate Form B, has an X-ray powder diffraction (XRPD) pattern comprising 2-theta peaks (2 theta±0.2) 10.6°, 11.4°, 15.8°, 16.6°, 19.2°, 20.2°, 21.1°, 23.3°, 24.0°, 24.3°, and 24.5°.

In some embodiments, a solid succinate salt form of mesembrine, such as Succinate Form B, has an X-ray powder diffraction (XRPD) pattern comprising 2-theta peaks according to Table 10 having a relative intensity of greater than 10%. In some embodiments, a solid succinate salt form of mesembrine, such as Succinate Form B, has an X-ray powder diffraction (XRPD) pattern according to FIG. 6A.

In some embodiments, a solid succinate salt form of mesembrine, such as Succinate Form B, is characterized by an X-ray powder diffraction (XRPD) having 2-theta peaks (2 theta±0.2) and corresponding d-spacing (angstroms±0.2):

| | |
|---|---|
| 20+0.2 [°] | d-spacing +0.2 |
| | [A] |
| 10.6 | 8.4 |
| 15.8 | 5.6 |
| 19.2 | 4.6 |
| 21.1 | 4.2 |
| 24.3 | 3.7 |

In some embodiments, a solid succinate salt form of mesembrine, such as Succinate Form B, is characterized by an X-ray powder diffraction (XRPD) having 2-theta peaks (2 theta±0.2) and corresponding d-spacing (angstroms±0.2):

| | |
|---|---|
| 20[°] | d-spacing [A] |
| 10.6 | 8.4 |
| 11.4 | 7.8 |
| 15.8 | 5.6 |
| 16.6 | 5.3 |
| 19.2 | 4.6 |
| 20.2 | 4.4 |
| 21.1 | 4.2 |
| 23.3 | 3.8 |
| 24.0 | 3.7 |
| 24.3 | 3.7 |
| 24.5 | 3.6 |

In some embodiments, a solid succinate salt form of mesembrine, such as Succinate Form B, is characterized by a TGA thermogram according to FIG. 6B. In some embodiments, a solid succinate salt form of mesembrine, such as Succinate Form B, is further characterized by a sample weight loss of up to about 5.1% upon heating up to 100° C. measured by TGA according to the following parameters:

| | |
|---|---|
| Parameters | TGA |
| Method | Ramp |
| Sample pan | Aluminum, open |
| Temperature | RT - 350 ° C. |
| Heating rate | 10 °C/min |
| Purge gas | N2 |

In some embodiments, a solid succinate salt form of mesembrine, such as Succinate Form B, is characterized by a differential scanning calorimetry (DSC) thermogram according to FIG. 6C. In some embodiments, a solid succinate salt form of mesembrine, such as Succinate Form B, is further characterized by a DSC having an endotherm at about 88° C. to about 103° C. (peak temperature), measured by DSC according to the following parameters:

| | |
|---|---|
| Parameters | DSC |
| Method | Ramp |
| Sample pan | Aluminum, crimped |
| Temperature | 25 ° C. - 230 ° C. |
| Heating rate | 10 ° C./min |
| Purge gas | N2 |

### Naphthalene Disulfonate Form A

In some embodiments, solid forms of a naphthalene disulfonate salt of Compound 1 are provided. Naphthalene Disulfonate Form A is a solid form of the naphthalene disulfonate salt of Compound 1 that can be identified by a XRPD pattern comprising certain characteristic peaks, and/or by one or more other techniques including DSC, TGA, and/or DVS.

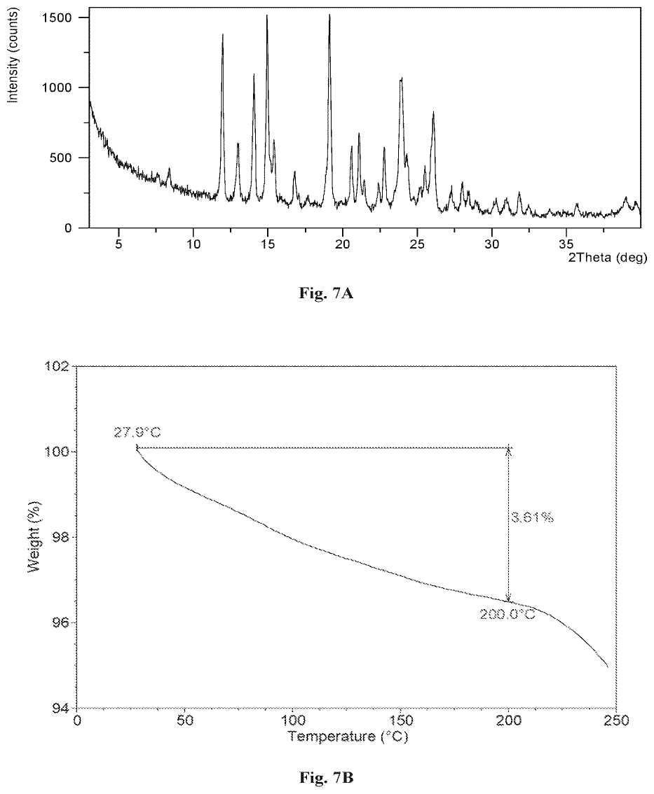

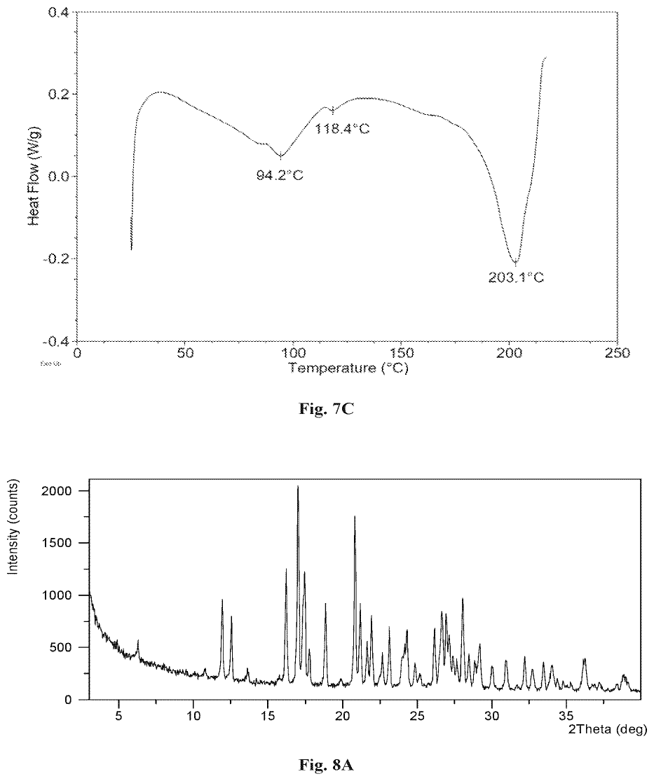

Samples of Naphthalene Disulfonate Form A were prepared according to the Example 8 and characterized by XRPD (e.g., a Pattern the same or substantially similar to or not dissimilar to FIG. 7A), TGA (e.g., a thermogram the same or substantially similar to or not dissimilar to FIG. 7B), and DSC (e.g., a thermogram the same or substantially similar to or not dissimilar to FIG. 7C).

In some embodiments, a solid naphthalene disulfonate salt form of mesembrine, such as Naphthalene Disulfonate Form A, has an X-ray powder diffraction (XRPD) pattern comprising 2-theta peaks (2 theta±0.2) 12.0°, 14.9°, 19.1°, 23.8°, and 24.0°. In some embodiments, a solid naphthalene disulfonate salt form of mesembrine, such as Naphthalene Disulfonate Form A, A has an X-ray powder diffraction (XRPD) pattern comprising 2-theta peaks (2 theta±0.2) 12.0°, 13.0°, 14.1°, 14.9°, 15.4°, 16.8°, 19.1°, 20.6°, 21.1°, 21.4°, 22.4°, 22.8°, 23.8°, 24.0°, 24.4°, 25.5°, 26.2°, 28.0°, 28.4°, and 31.8°.

In some embodiments, a solid naphthalene disulfonate salt form of mesembrine, such as Naphthalene Disulfonate Form A, has an X-ray powder diffraction (XRPD) pattern comprising 2-theta peaks according to Table 11 having a relative intensity of greater than 10%. In some embodiments, a solid naphthalene disulfonate salt form of mesembrine, such as Naphthalene Disulfonate Form A, has an X-ray powder diffraction (XRPD) pattern according to FIG. 7A.

In some embodiments, a solid naphthalene disulfonate salt form of mesembrine, such as Naphthalene Disulfonate Form A, is characterized by an X-ray powder diffraction (XRPD) having 2-theta peaks (2 theta±0.2) and corresponding d-spacing (angstroms±0.2):

| | |
|---|---|
| 20[°] | d-spacing [A] |
| 12.0 | 7.4 |
| 14.9 | 5.9 |
| 19.1 | 4.6 |
| 23.8 | 3.7 |
| 24.0 | 3.7. |

In some embodiments, a solid naphthalene disulfonate salt form of mesembrine, such as Naphthalene Disulfonate Form A, is characterized by an X-ray powder diffraction (XRPD) having 2-theta peaks (2 theta±0.2) and corresponding d-spacing (angstroms±0.2):

| | |
|---|---|
| 20[°] | d-spacing [A] |
| 12.0 | 7.4 |
| 13.0 | 6.8 |
| 14.1 | 6.3 |
| 14.9 | 5.9 |
| 15.4 | 5.8 |
| 16.8 | 5.3 |
| 19.1 | 4.6 |
| 20.6 | 4.3 |
| 21.1 | 4.2 |
| 21.4 | 4.1 |
| 22.4 | 4.0 |
| 22.8 | 3.9 |
| 23.8 | 3.7 |
| 24.0 | 3.7 |
| 24.4 | 3.7 |
| 25.5 | 3.5 |
| 26.2 | 3.4 |
| 28.0 | 3.2 |
| 28.4 | 3.1 |
| 31.8 | 2.8. |

In some embodiments, a solid naphthalene disulfonate salt form of mesembrine, such as Naphthalene Disulfonate Form A, is characterized by a TGA thermogram according to FIG. 7B. In some embodiments, a solid naphthalene disulfonate salt form of mesembrine, such as Naphthalene Disulfonate Form A, is further characterized by a sample weight loss of up to about 3.6% upon heating up to 200° C. measured by TGA according to the following parameters:

| | |
|---|---|
| Parameters | TGA |
| Method | Ramp |
| Sample pan | Aluminum, open |
| Temperature | RT - 350 ° C. |
| Heating rate | 10 ° C./min |
| Purge gas | N2. |

In some embodiments, a solid naphthalene disulfonate salt form of mesembrine, such as Naphthalene Disulfonate Form A, is characterized by a differential scanning calorimetry (DSC) thermogram according to FIG. 7C. In some embodiments, a solid naphthalene disulfonate salt form of mesembrine, such as Naphthalene Disulfonate Form A, is further characterized by a DSC having an endotherm at about 92° C. to about 97° C., and/or at about 116° C. to about 121° C., and/or at about 200° C. to about 205° C. (peak temperature), measured by DSC according to the following parameters:

| | |
|---|---|
| Parameters | DSC |
| Method | Ramp |
| Sample pan | Aluminum, crimped |
| Temperature | 25 ° C. - 230 °° C. |
| Heating rate | 10 ° C./min |
| Purge gas | N2. |

### HBr Form A

In some embodiments, solid forms of an HBr salt of Compound 1 are provided. HBr Form A is a solid form of the HBr salt of Compound 1 that can be identified by a XRPD pattern comprising certain characteristic peaks, and/or by one or more other techniques including DSC, TGA, and/or DVS.

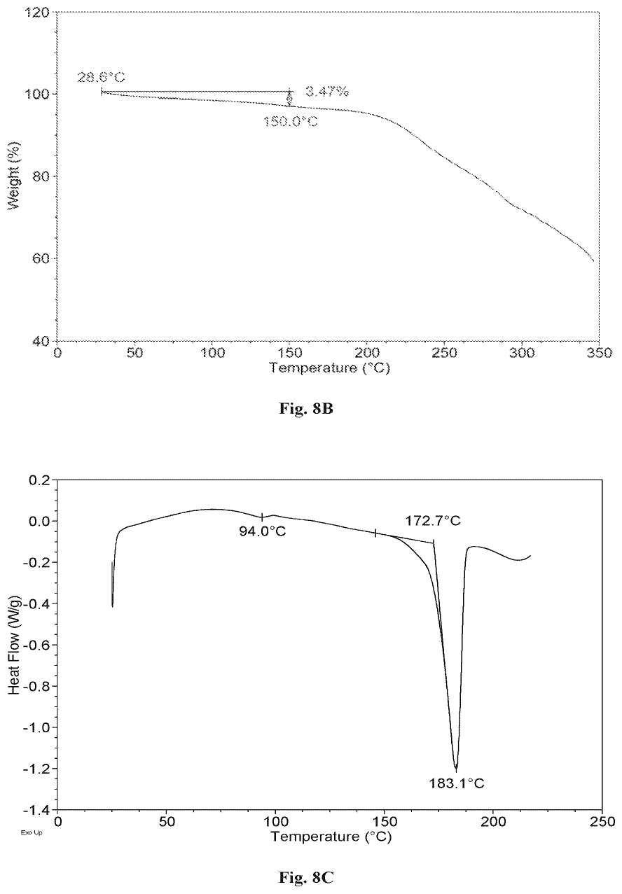

Samples of HBr Form A were prepared according to the Example 9 and characterized by XRPD (e.g., a Pattern the same or substantially similar to or not dissimilar to FIG. 8A), TGA (e.g., a thermogram the same or substantially similar to or not dissimilar to FIG. 8B), and DSC (e.g., a thermogram the same or substantially similar to or not dissimilar to FIG. 8C).

In some embodiments, a solid HBr salt form of mesembrine, such as HBr Form A, has an X-ray powder diffraction (XRPD) pattern comprising 2-theta peaks (2 theta±0.2) 16.2°, 17.0°, 17.5°, 21.2°, and 28.0°. In some embodiments, a solid HBr salt form of mesembrine, such as HBr Form A, has an X-ray powder diffraction (XRPD) pattern comprising 2-theta peaks (2 theta±0.2) 12.0°, 12.6°, 16.2°, 17.0°, 17.5°, 17.8°, 18.9°, 20.8°, 21.2°, 21.6°, 21.9°, 22.7°, 23.1°, 24.0°, 24.3°, 24.9°, 26.2°, 26.6°, 26.9°, 27.2°, 27.4°, 27.6°, 28.0°, 28.5°, 28.8°, 29.2°, 30.0°, 30.9°, 32.2°, 32.7°, 33.5°, 34.0°, and 36.2°.

In some embodiments, a solid HBr salt form of mesembrine, such as HBr Form A, has an X-ray powder diffraction (XRPD) pattern comprising 2-theta peaks according to Table 12 having a relative intensity of greater than 10%. In some embodiments, a solid HBr salt form of mesembrine, such as HBr Form A, has an X-ray powder diffraction (XRPD) pattern according to FIG. 8A.

In some embodiments, a solid HBr salt form of mesembrine, such as HBr Form A, is characterized by an X-ray powder diffraction (XRPD) having 2-theta peaks (2 theta±0.2) and corresponding d-spacing (angstroms±0.2):

| | |
|---|---|
| 20[°] | d-spacing [A] |
| 16.2 | 5.5 |
| 17.0 | 5.2 |
| 17.5 | 5.1 |
| 21.2 | 4.2 |
| 28.0 | 3.2. |

In some embodiments, a solid HBr salt form of mesembrine, such as HBr Form A, is characterized by an X-ray powder diffraction (XRPD) having 2-theta peaks (2 theta±0.2) and corresponding d-spacing (angstroms±0.2):

| 2θ[°] | d-spacing [Å] |
|---|---|
| 12.0 | 7.4 |
| 12.6 | 7.0 |
| 16.2 | 5.5 |
| 17.0 | 5.2 |
| 17.5 | 5.1 |
| 17.8 | 5.0 |
| 18.9 | 4.7 |
| 20.8 | 4.3 |
| 21.2 | 4.2 |
| 21.6 | 4.1 |
| 21.9 | 4.0 |
| 22.7 | 3.9 |
| 23.1 | 3.8 |
| 24.0 | 3.7 |
| 24.3 | 3.7 |
| 24.9 | 3.6 |
| 26.2 | 3.4 |
| 26.6 | 3.4 |
| 26.9 | 3.3 |
| 27.2 | 3.3 |
| 27.4 | 3.3 |
| 27.6 | 3.2 |
| 28.0 | 3.2 |
| 28.5 | 3.1 |
| 28.8 | 3.1 |
| 29.2 | 3.1 |
| 30.0 | 3.0 |
| 30.9 | 2.9 |
| 32.2 | 2.8 |
| 32.7 | 2.7 |
| 33.5 | 2.7 |
| 34.0 | 2.6 |
| 36.2 | 2.5. |

In some embodiments, a solid HBr salt form of mesembrine, such as HBr Form A, is characterized by a TGA thermogram according to FIG. 8B. In some embodiments, a solid HBr salt form of mesembrine, such as HBr Form A, is further characterized by a sample weight loss of up to about 3.5% upon heating up to 150° C. measured by TGA according to the following parameters:

| Parameters | TGA | |
|---|---|---|
| Method | Ramp | |
| Sample pan | Aluminum, open | |
| Temperature | RT-350° | C. |
| Heating rate | 10° | C./min |

| | |
|---|---|
| Purge gas | N₂. |

In some embodiments, a solid HBr salt form of mesembrine, such as HBr Form A, is characterized by a differential scanning calorimetry (DSC) thermogram according to FIG. 8C. In some embodiments, a solid HBr salt form of mesembrine, such as HBr Form A, is further characterized by a DSC having an endotherm at about 92° C. to about 97° C., and/or at about 180° C. to about 185° C. (peak temperature), measured by DSC according to the following parameters:

| | |
|---|---|
| Parameters | DSC |
| Method | Ramp |
| Sample pan | Aluminum, crimped |
| Temperature | 25° C.-230° C. |
| Heating rate | 10° C./min |
| Purge gas | N₂. |

In some embodiments, a solid form is produced by a process comprising:
1.) dissolving a free base of (−) mesembrine in acetone to form a (−) mesembrine solution;
2.) adding tartaric acid to the (−) mesembrine solution; and
3.) isolating the solids formed in step (2), wherein the isolated solids:
a) show a weight loss of about 1.4% to about 1.9% up to 130° C. by TGA; and/or
b) show an endotherm at about 150° C. to about 155° C. (peak temperature) by DSC.

In some embodiments, a solid form is produced by a process comprising:
1.) dissolving a free base of (−) mesembrine in ethanol to form a (−) mesembrine solution;
2.) adding fumaric acid to the (−) mesembrine solution; and
3.) isolating the solids formed in step (2), wherein the isolated solids:
a) show a weight loss of about 8.1% to about 8.6% up to 150° C. by TGA; and/or
b) show an endotherm at about 92° C. to about 97° C. (peak temperature) and/or an endotherm at about 145° C. to about 150° C. (peak temperature) (peak temperature) by DSC.

In some embodiments, a solid form is produced by a process comprising:
1.) dissolving a free base of (−) mesembrine in ethyl acetate to form a (−) mesembrine solution;
2.) adding malic acid to the (−) mesembrine solution; and
3.) isolating the solids formed in step (2),
   wherein the isolated solids:
a) show a weight loss of about 8.2% to about 8.7% up to 100° C. by TGA; and/or
b) show an endotherm at about 70° C. to about 75° C. (peak temperature) by DSC.

In some embodiments, a solid form is produced by a process comprising:
1.) dissolving a free base of (−) mesembrine in acetone to form a (−) mesembrine solution;
2.) adding malic acid to the (−) mesembrine solution;
3.) adding n-heptane to the solution formed in step (2); and
4.) isolating the solids formed in step (3),
   wherein the isolated solids:
a) show a weight loss of about 5.5% to about 6.0% up to 100° C. by TGA; and/or
b) show an endotherm at about 70° C. to about 75° C. (peak temperature) by DSC.

In some embodiments, a solid form is produced by a process comprising:
1.) dissolving a free base of (−) mesembrine in ethyl acetate to form a (−) mesembrine solution;
2.) adding succinic acid to the (−) mesembrine solution; and
3.) isolating the solids formed in step (2), wherein the isolated solids:
a) show a weight loss of about 4.7% to about 5.2% up to 100° C. by TGA; and/or
b) show an endotherm at about 64° C. to about 69° C. (peak temperature) by DSC.

In some embodiments, a solid form is produced by a process comprising:
1.) dissolving a free base of (−) mesembrine in methyl tert-butyl ether to form a (−) mesembrine solution;
2.) adding succinic acid to the (−) mesembrine solution; and
3.) isolating the solids formed in step (2),
   wherein the isolated solids:
a) show a weight loss of about 4.9% to about 5.4% up to 100° C. by TGA; and/or
b) show an endotherm at about 88° C. to about 103° C. (peak temperature) by DSC.

In some embodiments, a solid form is produced by a process comprising:
1.) dissolving a free base of (−) mesembrine in ethanol to form a (−) mesembrine solution;
2.) adding naphthalene-1,5-disulfonic acid to the (−) mesembrine solution; and
3.) isolating the solids formed in step (2),
   wherein the isolated solids:
a) show a weight loss of about 3.4% to about 3.9% up to 200° C. by TGA; and/or
b) show an endotherm at about 92° C. to about 97° C., and/or at about 116° C. to about 121° C., and/or at about 200° C. to about 205° C. (peak temperature) by DSC.

In some embodiments, a solid form is produced by a process comprising:
1.) dissolving a free base of (−) mesembrine in acetone to form a (−) mesembrine solution;
2.) adding hydrobromic acid to the (−) mesembrine solution; and
3.) adding n-heptane to the solution formed in step (2); and
4.) isolating the solids formed in step (3),
   wherein the isolated solids:
a) show a weight loss of about 3.2% to about 3.7% up to 130° C. by TGA; and/or
b) show an endotherm at about 92° C. to about 97° C., and/or at about 180° C. to about 185° C. (peak temperature) by DSC.

### Pharmaceutical Compositions & Methods of Treatment

In some embodiments, pharmaceutical compositions comprising mesembrine, and pharmaceutically acceptable salts and hydrates thereof are provided. The pharmaceutical composition can comprise mesembrine in one or more solid forms provided herein, such as crystalline mesembrine in a hydrated or anhydrous solid form. A pharmaceutical composition, as used herein, refers to a mixture of mesembrine optionally further comprising other pharmaceutically acceptable components, such as carriers, stabilizers, diluents, dispersing agents, suspending agents, thickening agents, and/or excipients. The pharmaceutical composition can facilitate administration of the compound to a mammal, including compositions formulated for oral administration of mesembrine to a mammal (e.g., capsules or tablets).

In some embodiments, crystalline mesembrine is incorporated into pharmaceutical compositions to provide solid oral dosage forms. In other embodiments, crystalline mesembrine is used to prepare pharmaceutical compositions prepared for oral solid dosage forms. In some embodiments, the pharmaceutical composition comprises an active pharmaceutical ingredient (API) comprising, consisting essentially of, or consisting of mesembrine prepared under applicable Good Manufacturing Practice (GMP). For example, the pharmaceutical composition can be a batch composition comprising mesembrine, wherein the batch composition adheres to Good Manufacturing Practices (e.g., ICH Harmonised Tripartite Guideline, Good Manufacturing Practice Guide for Active Pharmaceutical Ingredients Q7, Current Step 4 version dated 10 Nov. 2010). More preferably, the GMP batch composition can be a homogenous blended batch comprising mesembrine. The FDA (Food and Drug Administration) provides applicable guidance on Good Manufacturing Practice (GMP) for the manufacturing of active pharmaceutical ingredients (APIs) under an appropriate system for managing quality. As used with respect to manufacture of API under GMP, “manufacturing” is defined to include all operations of receipt of materials, production, packaging, repackaging, labelling, relabelling, quality control, release, storage and distribution of APIs and the related controls. An “API Starting Material” is a raw material, intermediate, or an API that is used in the production of an API and that is incorporated as a significant structural fragment into the structure of the API. An API Starting Material can be an article of commerce, a material purchased from one or more suppliers under contract or commercial agreement, or produced in-house. API Starting Materials normally have defined chemical properties and structure.

The pharmaceutical compositions comprising mesembrine can be administered to patients in need thereof, to provide a therapeutically effective amount of a compound of mesembrine.

In practicing the methods of treatment or use provided herein, therapeutically effective amounts of mesembrine are administered in a pharmaceutical composition to a mammal having a disease, disorder, or condition to be treated. In some embodiments, the disease, disorder, or condition is a central nervous system disorder or an inflammatory condition. In some embodiments, pharmaceutical compositions reported herein can be provided in a unit dosage form container (e.g., in a vial or bag or the like).

In some embodiments, methods of treating a patient suffering from a disease comprise administering to a patient a composition comprising a compound disclosed herein for the treatment or prevention of a mental health disorder. In some embodiments, methods of treating a patient suffering from a disease comprise administering to a patient a composition comprising a compound disclosed herein for the treatment or prevention of a diagnosed condition selected from anxiety and depression. In some embodiments, the compound disclosed herein is administered to the patient in a unit dose. In some embodiments, a method comprises the administration to a patient in need thereof of a therapeutically effective amount of a mesembrine composition for the treatment of a disease selected from the group consisting of mild to moderate depression and major depressive episodes. In some embodiments, a method comprises the administration to a patient in need thereof of a therapeutically effective amount of a mesembrine composition for the treatment of anxiety. In some embodiments, a method comprises the administration to a patient in need thereof of a therapeutically effective amount of a mesembrine composition for the treatment of depression. In some embodiments, a method comprises the administration to a patient in need thereof of a therapeutically effective amount of a mesembrine composition for the treatment of a condition selected from the group consisting of: anxiety associated with depression, anxiety with depression, mixed anxiety and depressive disorder. In some embodiments, a method comprises the administration to a patient in need thereof of a therapeutically effective amount of a mesembrine composition for the treatment of anxiety and hysteria or anxiety and depression.

In some embodiments, pharmaceutical compositions reported herein can be provided in an oral dosage form. In some embodiments, an oral dosage form of mesembrine can be a capsule. In some embodiments, an oral dosage form of mesembrine is a tablet. In some embodiments, an oral dosage form comprises a filler. In some embodiments, an oral dosage form comprises two fillers. In some embodiments, an oral dosage form comprises one or more fillers. In some embodiments, an oral dosage form comprises one or more disintegrants. In some embodiments, the oral dosage form comprises one or more lubricants. In some embodiments, an oral dosage form comprises one or more glidants, anti-adherents and/or anti-statics. In some embodiments, an oral dosage form is prepared via dry blending. In some embodiments, an oral dosage form is a tablet and is prepared via dry granulation.

In some embodiments, a pharmaceutical composition comprises a solid form described herein; and a pharmaceutically acceptable excipient. In some embodiments, a pharmaceutical composition is formed by a process comprising dissolving a solid form described herein.

In some embodiments, a method of treating a mental health disorder, comprises administering to a mammal in need thereof an effective amount of a solid form described herein or a pharmaceutical composition described herein. In some embodiments, the mental health disorder is anxiety, stress, or depression. In some embodiments, the mammal is a human.

In some embodiments, a method of treating a mental health disorder, comprises administering to a mammal in need thereof an effective amount of a solid form of (−) mesembrine selected from:
a.) Tartrate Form B having an X-ray powder diffraction (XRPD) pattern comprising 2-theta peaks (2 theta±0.2) at 21.0°;
b.) Fumarate Form A having an X-ray powder diffraction (XRPD) pattern comprising 2-theta peaks (2 theta±0.2) at 17.3°;
c.) Malate Form A having an X-ray powder diffraction (XRPD) pattern comprising 2-theta peaks (2 theta±0.2) at 8.6°;
d.) Malate Form C having an X-ray powder diffraction (XRPD) pattern comprising 2-theta peaks (2 theta±0.2) at 8.4°;
e.) Succinate Form A having an X-ray powder diffraction (XRPD) pattern comprising 2-theta peaks (2 theta±0.2) at 24.2°;
f.) Succinate Form B having an X-ray powder diffraction (XRPD) pattern comprising 2-theta peaks (2 theta±0.2) at 15.8°;
g.) Naphthalene Disulfonate Form A having an X-ray powder diffraction (XRPD) pattern comprising 2-theta peaks (2 theta±0.2) at 19.1°; and
h.) HBr Form A having an X-ray powder diffraction (XRPD) pattern comprising 2-theta peaks (2 theta±0.2) at 17.0°.

In some embodiments, the recited XRPD peaks for each solid form are the most intense peaks by relative intensity in the respective XRPD patterns.

In some embodiments, the XRPD pattern of Tartrate Form B further comprises 2-theta peaks (2 theta±0.2) at 15.2°.

In some embodiments, the XRPD pattern of Malate Form A further comprises 2-theta peaks (2 theta±0.2) at 16.0°.

In some embodiments, the XRPD pattern of Tartrate Form B further comprises 2-theta peaks (2 theta±0.2) at 14.4°.

### Examples

### Instruments and Methods

### XRPD

For XRPD analysis, Empyrean X-ray powder diffractometer from Malvern Panalytical was used. Sample was spread on the middle of a zero-background Si holder. The XRPD parameters used are listed in Table 1.

TABLE 1
Parameters for XRPD test

| | |
|---|---|
| Parameters | Reflection Mode |
| Model | Empyrean |
| X-Ray wavelength | Cu, kα, |
| | Kα1 (Å): 1.540598, |
| | Kα2 (Å): 1.544426 |
| | Kα2/Kα1 intensity |
| | ratio: 0.50 |
| X-Ray tube setting | 45 kV, 40 mA |
| Divergence slit | ⅛° |
| Scan mode | Continuous |
| Scan range (°2TH) | 3°-40° |
| Scan step time (s) | 46.67 |
| Step size (°2TH) | 0.0263 |
| Test time | 5 min 4 s |

### TGA

TGA data were collected using a TA Discovery TGA 5500 TGA from TA Instruments. Detailed parameters used are listed in Table 2.

TABLE 2
Parameters for TGA test

| Parameters | TGA | |
|---|---|---|
| Method | Ramp | |
| Sample pan | Aluminum, open | |
| Temperature | RT-350° | C. |
| Heating rate | 10° | C./min |

| | |
|---|---|
| Purge gas | N₂ |

### DSC

DSC data were collected using a TA Discovery DSC 2500 DSC from TA Instruments. Detailed parameters used are listed in Table 3.

TABLE 3
Parameters for DSC test

| | |
|---|---|
| Parameters | DSC |
| Method | Ramp |
| Sample pan | Aluminum, crimped |
| Temperature | 25° C.-230° C. |
| Heating rate | 10° C./min |
| Purge gas | N₂ |

Purity HPLC Conditions

| Instrument | Agilent 1260 with DAD detector | |
|---|---|---|
| Column | Waters Xbridge C18, 4.6*150 mm, | |
| | 5 μm | |
| Mobile phase | A: 0.0375% TFA in H₂O | |
| | B: 0.01875% TFA in ACN | |
| Gradient | Time (min) | % B |
| | 0.0 | 5 |
| | 2.0 | 5 |
| | 12.0 | 30 |
| | 20.0 | 90 |
| | 22.0 | 90 |
| | 22.1 | 5 |
| | 28.0 | 5 |

| | |
|---|---|
| Flow rate | 1.0 mL/min |
| Injection volume | 5 μL |
| Detector wavelength | 230 nm |
| Column temperature | 40° C. |
| Sampler temperature | RT |
| Diluent | ACN/H₂O (1:1, v/v) |

Solubility HPLC Conditions

| Instrument | Agilent 1260 with DAD detector | |
|---|---|---|
| Column | Waters Xbridge C18, 4.6*150 mm, 5 | |
| | μm | |
| Mobile phase | A: 0.0375% TFA in H₂O | |
| | B: 0.01875% TFA in ACN | |
| Gradient | Time (min) | % B |
| | 0.0 | 5 |
| | 4.0 | 30 |
| | 6.0 | 90 |
| | 8.0 | 90 |
| | 8.1 | 5 |
| | 12.0 | 5 |

| | |
|---|---|
| Flow rate | 1.0 mL/min |
| Injection | 5 μL |
| volume | |
| Detector | 230 nm |
| wavelength | |
| Column | 40° C. |
| temperature | |
| Sampler | RT |
| temperature | |
| Diluent | ACN/H₂O (1:1, v/v) |

### Mesembrine Synthesis

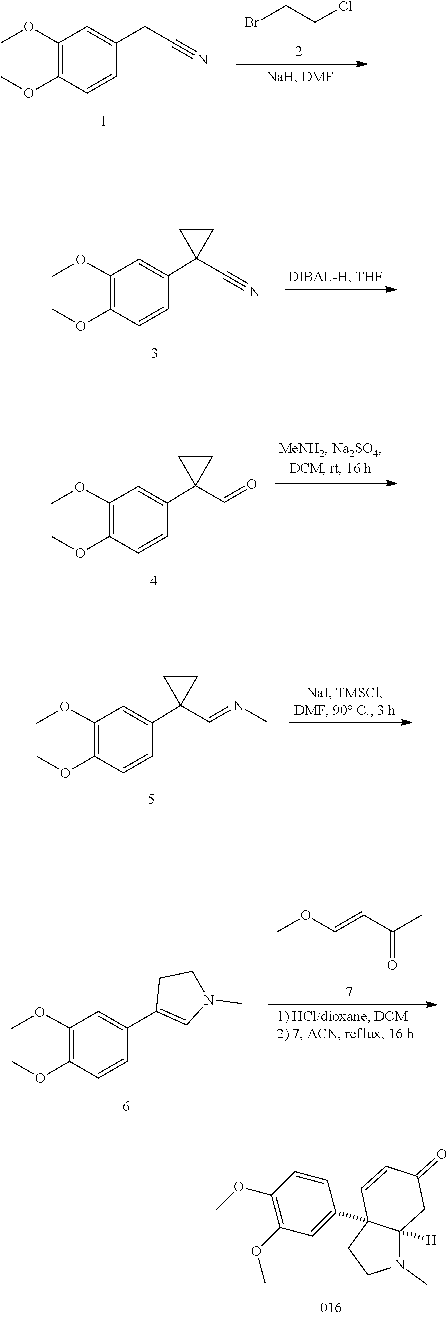

### Step 1-1-(3,4-dimethoxyphenyl)cyclopropanecarbonitrile (3)

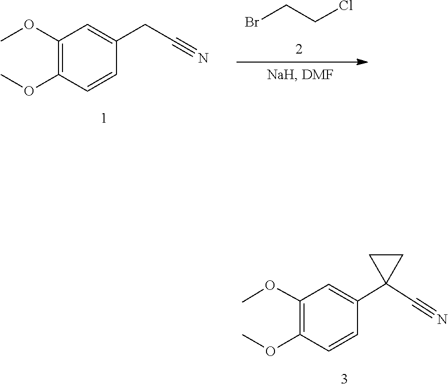

A mixture of 2-(3,4-dimethoxyphenyl) acetonitrile (20 g, 112 mmol, CAS #93-17-4) in DMF (93 mL) was added NaH (18.0 g, 451 mmol, 60% purity) in portions and the mixture was stirred at 25° C. for 20 minutes. 1-bromo-2-chloro-ethane (16.1 g, 112 mmol, CAS #107-04-0) was added, and then mixture stirred at 25° C. for 16 hours. On completion, the reaction was quenched by methanol/water mixture (1:1, 1000 mL) and the reaction products were extracted into ethyl acetate (3×500 mL). The combined extracts were washed with water (4×500 mL), brine (1×200 mL) and then dried (Na₂SO₄). The solvent was then removed under reduced pressure to give residue. The residue was purified by column chromatography (SiO₂, Petroleum ether/Ethyl acetate=10/1 to 3/1) to give the title compound (15 g, 65% yield) as yellow oil.

¹H NMR (400 MHz, CHLOROFORM-d) δ 6.88 (s, 1H), 6.82 (d, J=1.2 Hz, 2H), 3.91 (s, 3H), 3.88 (s, 3H), 1.68-1.65 (m, 2H), 1.35 (d, J=2.4 Hz, 2H).

### Step 2-1-(3,4-dimethoxyphenyl)cyclopropanecarbaldehyde (4)

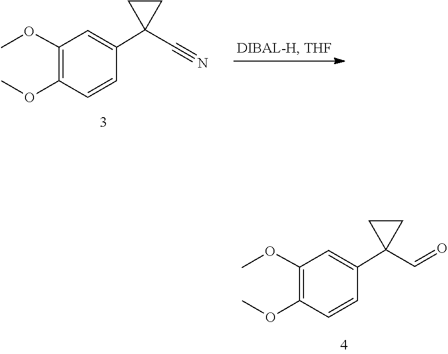

To a solution of 1-(3,4-dimethoxyphenyl)cyclopropanecarbonitrile (11 g, 54.1 mmol) in THF (160 mL) was added DIBAL-H (1 M, 81.19 mL). The mixture was stirred at 25° C. for 3 hours. On completion, the reaction was cautiously quenched by addition of 2 M HCl aqueous soliton, extracted with dichloromethane (3×200 mL). The combined extracts were washed with water (2×200 mL), brine (2×200 mL) and then dried (Na₂SO₄) to give the title compound (9.6 g, 85% yield) as yellow oil.

LC-MS (ESI+) m/z 207.0 (M+H)⁺

¹H NMR (400 MHz, CHLOROFORM-d) δ 9.26 (s, 1H), 6.94-6.61 (m, 3H), 3.89 (d, J=2.8 Hz, 6H), 1.61-1.52 (m, 2H), 1.42-1.37 (m, 2H)

### Step 3-(Z)-1-[1-(3,4-dimethoxyphenyl)cyclopropyl]-N-methyl-methanimine (5)

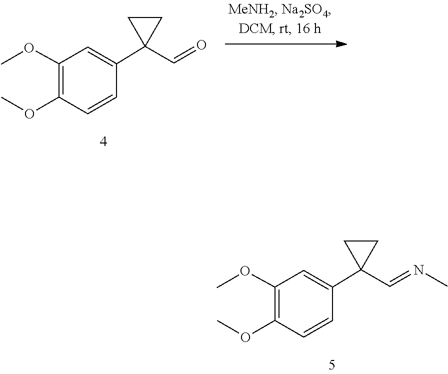

To a solution of 1-(3,4-dimethoxyphenyl)cyclopropanecarbaldehyde (5.0 g, 24.2 mmol) in DCM (50 mL) was added MeNH₂ (2 M, 121 mL) and Na₂SO₄ (15.5 g, 109 mmol, 11.0 mL). The mixture was stirred at 25° C. for 16 hours. On completion, the reaction mixture was filtered and the filtrate was concentrated under reduced pressure to give the title compound (5.1 g, 99% yield) as white solid. LC-MS (ESI⁺) m/z 219.9 (M+H)⁺, major Int.4 mass on LCMS;

¹H NMR (400 MHz, CHLOROFORM-d) δ 7.55 (q, J=1.2 Hz, 1H), 6.93-6.77 (m, 3H), 3.88 (d, J=7.2 Hz, 6H), 3.24 (d, J=1.6 Hz, 3H), 1.29-1.23 (m, 2H), 1.18-1.12 (m, 2H).

### Step 4-4-(3,4-dimethoxyphenyl)-1-methyl-2,3-dihydropyrrole (6)

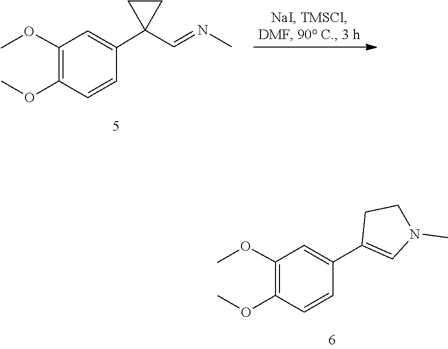

To a solution of (Z)-1-[1-(3,4-dimethoxyphenyl)cyclopropyl]-N-methyl-methanimine (5.4 g, 24.6 mmol) in DMF (19 mL) was added NaI (366 mg, 2.44 mmol) and TMSCl (267 mg, 2.46 mmol). The mixture was stirred at 90° C. for 3 hours. On completion, the reaction mixture was diluted with water (100 mL) and extracted with EtOAc (100 mL×3). The organic phase was wash with water and brine, dried over with anhydrous sodium sulfate, concentrated under reduced pressure to give the title compound (6.25 g, 80% yield) as yellow oil.

LC-MS (ESI⁺) m/z 220.0 (M+H)⁺.

¹H NMR (400 MHz, CHLOROFORM-d) δ 6.90-6.66 (m, 3H), 6.31 (t, J=1.6 Hz, 1H), 3.95-3.80 (m, 6H), 3.18-3.11 (m, 2H), 2.79 (dt, J=1.2, 9.0 Hz, 2H), 2.65 (s, 3H).

### Step 5-3a-(3,4-dimethoxyphenyl)-1-methyl-2,3,7,7a-tetrahydroindol-6-one (016)

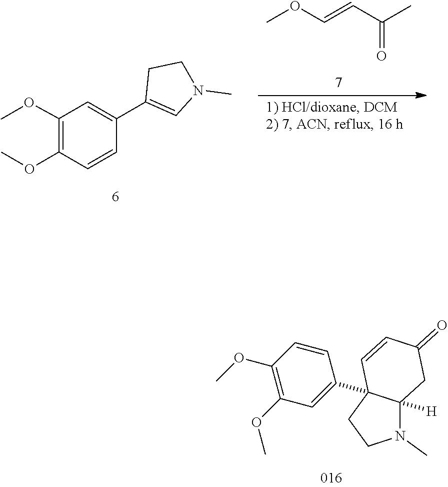

4-(3,4-dimethoxyphenyl)-1-methyl-2,3-dihydropyrrole (6.25 g, 28.5 mmol) was dissolved in dichloromethane (100 mL). To this was added HCl/dioxane (25 mL of a 1.0 M solution, 100 mmol) and the crude HCl salt was evaporated to dryness. A solution of obtained HCl slat, (E)-4-methoxybut-3-en-2-one (4.28 g, 42.7 mmol, CAS #4652-27-1) in ACN (90 mL) was stirred at 90° C. for 16 hours. On completion, the reaction mixture was filtered and the filtrate was concentrated under reduced pressure to give a residue, the residue was purified by prep-HPLC (column: Phenomenex luna C18 (250*70 mm, 10 um); mobile phase: [water (NH₄HCO₃)-ACN]; B %: 22%-52%,20 min) and acidified with aq. HCl to give desired compound (3.0 g, 30% yield) as a white solid.

LC-MS (ESI⁺) m/z 288.3 (M+H)⁺.

¹H NMR (400 MHz, CHLOROFORM-d) δ 6.90-6.88 (m, 1H), 6.87-6.83 (m, 2H), 6.74 (dd, J=2.0, 10.1 Hz, 1H), 6.11 (d, J=10.0 Hz, 1H), 3.89 (d, J=4.0 Hz, 6H), 3.33 (dt, J=2.4, 8.8 Hz, 1H), 2.69-2.66 (m, 1H), 2.58-2.51 (m, 2H), 2.50-2.41 (m, 2H), 2.33 (s, 3H), 2.27-2.18 (m, 1H)

### Step 6 (3aR,7aR)-3a-(3,4-dimethoxyphenyl)-1-methyloctahydro-6H-indol-6-one (022)

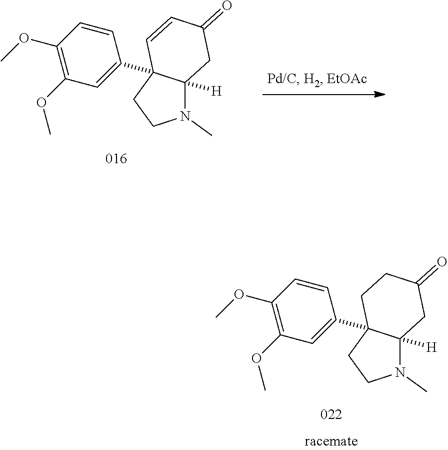

To a mixture of 3a-(3,4-dimethoxyphenyl)-2,3,7,7a-tetrahydro-1H-indol-6-one (12.0 g, 43.9 mmol, 016), Pd/C (300 mg, 4.39 mmol, 10% purity) in EtOAc (120 mL) was degassed and purged with H₂ for 3 times, and then the mixture was stirred at 25° C. for 2 hours under H₂ atmosphere (15 psi). On completion, the reaction mixture was filtered and concentrated in vacuo to give the title compound (10 g, 80% yield) as brown oil.

LC-MS (ESI⁺) m/z 290.4 (M+H)⁺

¹H NMR (400 MHZ, CDCl₃) δ 6.99-6.89 (m, 2H), 6.89-6.84 (m, 1H), 3.91 (d, J=7.6 Hz, 6H), 3.20-3.11 (m, 1H), 2.97 (t, J=3.6 Hz, 1H), 2.69-2.56 (m, 2H), 2.51-2.31 (m, 5H), 2.27-2.18 (m, 3H), 2.18-2.07 (m, 2H).

### Step 6 Chiral Resolution (001)

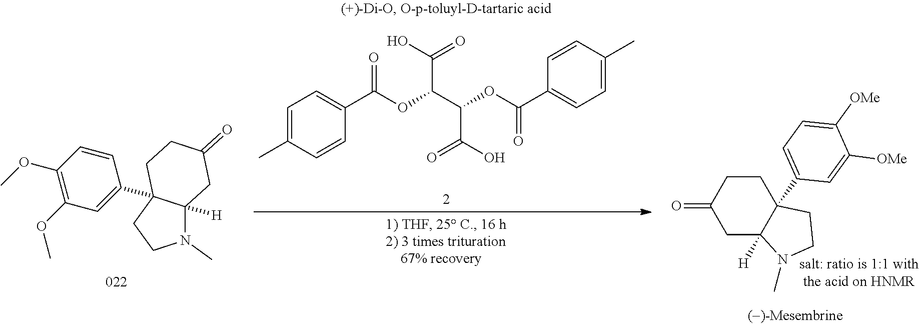

To a solution of 3a-(3,4-dimethoxyphenyl)-1-methyl-2,3,4,5,7,7a-hexahydroindol-6-one (28.0 g, 85.1 mmol) in THF (1400 mL, 50 V) was added (2S,3S)-2,3-bis[(4-methylbenzoyl)oxy]butanedioic acid (19.7 g, 51.1 mmol, CAS: 32634-68-7). The obtained suspension was stirred at 25° C. for 16 hours and then filtered. The solid was dried under vacuo and collected to give (3aS,7aS)-3a-(3,4-dimethoxyphenyl)-1-methyl-2,3,4,5,7,7a-hexahydroindol-6-one (25.0 g, 98% purity, 72% de, salt with acid 2) as a white solid. Then the obtained solid was triturated with THF (30 V each for 3 times) at 25° C. for 16 hours to give (3aS,7aS)-3a-(3,4-dimethoxyphenyl)-1-methyl-2,3,4,5,7,7a-hexahydroindol-6-one (22 g, 98% purity, 95% de, salt with acid 2) as a white solid.

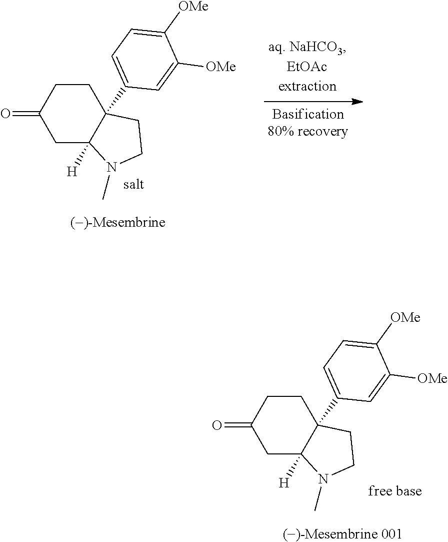 [SMILES: COc1ccc([C@@]23CCC(=O)C[C@@H]2N(C)CC3)cc1OC ; COc1ccc([C@@]23CCC(=O)C[C@@H]2N(C)CC3)cc1OC]

(3aS,7aS)-3a-(3,4-dimethoxyphenyl)-1-methyl-2,3,4,5,7,7a-hexahydroindol-6-one (22 g, salt) was poured to the saturated sodium bicarbonate solution (500 mL) and extracted with ethyl acetate (500 mL). The organic layers was dried by sodium sulfate, filtered and concentrated in vacuo to give the (3aS,7aS)-3a-(3,4-dimethoxyphenyl)-1-methyl-2,3,4,5,7,7a-hexahydroindol-6-one (7.50 g, 95% purity, 95% ee, free base) as a yellow gum.

LC-MS (ESI⁺) m/z 290.6 (M+H)⁺

¹H NMR (400 MHZ, CDCl₃) δ 6.89-6.81 (m, 2H), 6.80-6.75 (m, 1H), 3.82 (d, J=8.0 Hz, 6H), 3.11-3.03 (m, 1H), 2.88 (t, J=3.6 Hz, 1H), 2.59-2.48 (m, 2H), 2.43-2.32 (m, 1H), 2.31-2.21 (m, 4H), 2.20-2.09 (m, 3H), 2.08 (br s, 2H).

### Example 1—Freeform Form A

Freeform Form A was a gum material. The material had the solubility shown below:

| Solvent | Solubility (mg/mL) | Solvent | Solubility (mg/mL) |
|---|---|---|---|
| MeOH | S > 36.0 | THF | S > 44.0 |
| EtOH | S > 42.0 | 2-MeTHF | S > 40.0 |
| IPA | S > 38.0 | CPME | S > 40.0 |
| Acetone | S > 40.0 | 1,4-Dioxane | S > 40.0 |
| MIBK | S > 38.0 | ACN | S > 44.0 |
| EtOAc | 19.0 < S < 38.0 | DCM | S > 42.0 |
| IPAc | S > 36.0 | DMSO | S > 42.0.0 |
| MTBE | S > 40.0 | H₂O | S > 42.0 |

The solid was analyzed by XRPD, TGA, and DSC as described above. By TGA, the sample showed weight loss of 3.14% up to 150° C. DSC showed a broad endotherm at 69.6° C. (peak temperature).

### Table 4 Diffraction Peaks of Freeform Form A:

| #Peak | 2θ[°] | d-spacing [Å] | Rel. Int. [%] |
|---|---|---|---|
| 1 | 7.45 | 11.87 | 14.31 |
| 2 | 12.90 | 6.86 | 50.95 |
| 3 | 13.93 | 6.36 | 39.61 |
| 4 | 14.91 | 5.94 | 40.25 |
| 5 | 17.47 | 5.08 | 13.79 |
| 6 | 19.02 | 4.67 | 22.66 |
| 7 | 19.75 | 4.49 | 100.00 |
| 8 | 22.42 | 3.97 | 6.69 |
| 9 | 23.04 | 3.86 | 37.12 |
| 10 | 24.78 | 3.59 | 4.34 |
| 11 | 25.38 | 3.51 | 23.36 |
| 12 | 25.94 | 3.43 | 7.02 |
| 13 | 26.99 | 3.30 | 11.84 |
| 14 | 28.55 | 3.13 | 7.80 |
| 15 | 29.54 | 3.02 | 7.01 |
| 16 | 30.03 | 2.98 | 7.06 |
| 17 | 32.78 | 2.73 | 6.31 |
| 18 | 34.93 | 2.57 | 6.26 |
| 19 | 36.60 | 2.46 | 2.88 |

### Example 2—Tartrate Form B

### Preparation Method:

1. Freeform Form A (560.1 mg) was weighed into a 20 mL glass vial. Acetone (14 mL) was added to dissolve Freeform Form A solids.
2. L-tartaric acid (10.6 mg) was weighed into an HPLC vial. Acetone solution of Freeform Form A (0.5 mL) was added into the vial.
3. The mixture was magnetically stirred at room temperature (RT) for 3 days.
4. The suspension was centrifuged (10000 rpm, 2 min) and solids were dried at RT under vacuum for 1 day.

The resulting solid was analyzed by XRPD, TGA, and DSC as described above. The results are shown in FIGS. 1A-1C. By TGA, the sample showed weight loss of 1.62% up to 130° C. DSC showed an endotherm at 152.7° C. (peak temperature).

TABLE 5
Diffraction peaks of Tartrate salt Form B

| #Peak | 2θ[°] | d-spacing [Å] | Rel. Int. [%] |
|---|---|---|---|
| 1 | 5.24 | 16.87 | 45.65 |
| 2 | 10.47 | 8.45 | 78.79 |
| 3 | 11.28 | 7.84 | 11.93 |
| 4 | 11.95 | 7.40 | 17.06 |
| 5 | 12.36 | 7.16 | 25.95 |
| 6 | 14.75 | 6.00 | 35.00 |
| 7 | 15.18 | 5.84 | 95.65 |
| 8 | 15.70 | 5.64 | 14.06 |
| 9 | 16.24 | 5.46 | 21.44 |
| 10 | 16.74 | 5.30 | 49.67 |
| 11 | 16.99 | 5.22 | 31.31 |
| 12 | 17.27 | 5.13 | 21.70 |
| 13 | 18.28 | 4.85 | 48.32 |
| 14 | 19.39 | 4.58 | 15.30 |
| 15 | 19.90 | 4.46 | 20.97 |
| 16 | 20.73 | 4.29 | 43.18 |
| 17 | 21.05 | 4.22 | 100.00 |
| 18 | 21.88 | 4.06 | 3.52 |
| 19 | 23.38 | 3.80 | 34.32 |
| 20 | 23.80 | 3.74 | 12.52 |
| 21 | 25.67 | 3.47 | 10.64 |
| 22 | 26.38 | 3.38 | 4.95 |
| 23 | 27.44 | 3.25 | 13.57 |
| 24 | 28.70 | 3.11 | 7.86 |
| 25 | 29.51 | 3.03 | 5.53 |
| 26 | 30.62 | 2.92 | 7.63 |
| 27 | 34.29 | 2.62 | 3.23 |
| 28 | 35.92 | 2.50 | 3.96 |

### Example 3—Fumarate Form A

### Preparation Method:

1. Freeform Form A (560.1 mg) was weighed into a 20 mL glass vial. Ethanol (14 mL) was added to dissolve Freeform Form A solids.
2. Fumaric acid (8.1 mg) was weighed into an HPLC vial. Ethanol solution of Freeform Form A (0.5 mL) was added into the vial.
3. The mixture was magnetically stirred at room temperature (RT) for 3 days.
4. The suspension was centrifuged (10000 rpm, 2 min) and solids were dried at RT under vacuum for 1 day.

The resulting solid was analyzed by XRPD, TGA, and DSC as described above. The results are shown in FIGS. 2A-2C. By TGA, the sample showed weight loss of 8.31% up to 150° C. DSC showed an endotherm at 95.7° C. and 146.8° C. (peak temperature).

TABLE 6
Diffraction peaks of Fumarate salt Form A

| #Peak | 2θ[°] | d-spacing [Å] | Rel. Int. [%] |
|---|---|---|---|
| 1 | 9.10 | 9.71 | 89.59 |
| 2 | 10.23 | 8.65 | 30.89 |
| 3 | 11.49 | 7.70 | 29.35 |
| 4 | 13.17 | 6.72 | 41.00 |
| 5 | 14.26 | 6.21 | 6.49 |
| 6 | 15.28 | 5.80 | 22.66 |
| 7 | 17.00 | 5.22 | 89.86 |
| 8 | 17.31 | 5.12 | 100.00 |
| 9 | 19.15 | 4.63 | 6.35 |
| 10 | 19.77 | 4.49 | 54.00 |
| 11 | 21.07 | 4.22 | 26.62 |
| 12 | 22.09 | 4.02 | 20.42 |
| 13 | 22.60 | 3.93 | 12.54 |
| 14 | 23.06 | 3.86 | 21.45 |
| 15 | 23.68 | 3.76 | 32.07 |
| 16 | 24.21 | 3.68 | 49.47 |
| 17 | 24.58 | 3.62 | 14.75 |
| 18 | 25.16 | 3.54 | 11.99 |
| 19 | 25.72 | 3.46 | 15.06 |
| 20 | 26.45 | 3.37 | 30.49 |
| 21 | 28.85 | 3.09 | 16.46 |
| 22 | 29.71 | 3.01 | 5.32 |
| 23 | 30.99 | 2.89 | 6.76 |
| 24 | 34.81 | 2.58 | 3.29 |

### Example 4—Malate Form A

### Preparation Method:

1. Freeform Form A (560.3 mg) was weighed into a 20 mL glass vial. Ethyl acetate (14 mL) was added to dissolve Freeform Form A solids.
2. L-malic acid (9.3 mg) was weighed into an HPLC vial. Ethyl acetate solution of Freeform Form A (0.5 mL) was added into the vial.
3. The mixture was magnetically stirred at room temperature (RT) for 3 days.
4. The suspension was centrifuged (10,000 rpm, 2 min) and solids were dried at RT under vacuum for 1 day.

The resulting solid was analyzed by XRPD, TGA, and DSC as described above. The results are shown in FIGS. 3A-3C. By TGA, the sample showed weight loss of 8.41% up to 100° C. DSC showed an endotherm at 72.3° C. (peak temperature).

TABLE 7
Diffraction peaks of Malate salt Form A

| #Peak | 2θ[°] | d-spacing [Å] | Rel. Int. [%] |
|---|---|---|---|
| 1 | 6.49 | 13.61 | 8.52 |
| 2 | 8.60 | 10.28 | 100.00 |
| 3 | 10.28 | 8.61 | 11.06 |
| 4 | 11.99 | 7.38 | 1.18 |
| 5 | 12.59 | 7.03 | 6.85 |
| 6 | 13.26 | 6.68 | 2.66 |
| 7 | 14.41 | 6.15 | 9.33 |
| 8 | 15.26 | 5.81 | 10.26 |
| 9 | 15.49 | 5.72 | 8.11 |
| 10 | 15.95 | 5.56 | 81.57 |
| 11 | 16.28 | 5.44 | 9.28 |
| 12 | 17.15 | 5.17 | 4.61 |
| 13 | 19.16 | 4.63 | 3.57 |
| 14 | 19.55 | 4.54 | 19.07 |
| 15 | 19.74 | 4.50 | 18.11 |
| 16 | 20.01 | 4.44 | 4.45 |
| 17 | 20.63 | 4.31 | 6.91 |
| 18 | 20.80 | 4.27 | 7.95 |
| 19 | 21.15 | 4.20 | 9.75 |
| 20 | 22.06 | 4.03 | 2.70 |
| 21 | 22.85 | 3.89 | 5.23 |
| 22 | 23.45 | 3.79 | 4.05 |
| 23 | 24.24 | 3.67 | 17.60 |
| 24 | 24.90 | 3.58 | 9.65 |
| 25 | 25.33 | 3.52 | 17.96 |
| 26 | 25.69 | 3.47 | 4.47 |
| 27 | 26.18 | 3.40 | 1.99 |
| 28 | 26.72 | 3.34 | 3.93 |
| 29 | 27.46 | 3.25 | 2.71 |
| 30 | 28.49 | 3.13 | 3.33 |
| 31 | 29.14 | 3.07 | 1.01 |
| 32 | 29.84 | 2.99 | 2.94 |
| 33 | 30.37 | 2.94 | 1.60 |
| 34 | 30.79 | 2.90 | 1.17 |
| 35 | 31.60 | 2.83 | 0.86 |
| 36 | 32.89 | 2.72 | 3.77 |
| 37 | 33.84 | 2.65 | 1.15 |
| 38 | 35.32 | 2.54 | 1.06 |
| 39 | 35.80 | 2.51 | 1.02 |
| 40 | 36.74 | 2.45 | 1.74 |
| 41 | 38.43 | 2.34 | 0.90 |
| 42 | 39.08 | 2.30 | 0.78 |

### Example 5—Malate Form C

### Preparation Method:

1. Freeform Form A (560.1 mg) was weighed into a 20 mL glass vial. Acetone (14 mL) was added to dissolve Freeform Form A solids.
2. L-malic acid (9.5 mg) was weighed into an HPLC vial. Acetone solution of Freeform Form A (0.5 mL) was added into the vial.
3. The mixture was magnetically stirred at room temperature (RT) for 2 days, then stirred at 5° C. for 1 day and −20° C. for 3 days.
4. Anti-solvent n-heptane (0.4 mL) was added into the solution. Gel-like sample was obtained.
5. The mixture was stirred with temperature cycling between 5° C. and 50° C. (3 cycles).
6. The suspension was centrifuged (10,000 rpm, 2 min) and solids were dried at RT under vacuum for 1 day.

The resulting solid was analyzed by XRPD, TGA, and DSC as described above. The results are shown in FIGS. 4A-4C. By TGA, the sample showed weight loss of 5.77% up to 100° C. DSC showed an endotherm at 72.0° C. (peak temperature).

TABLE 8
Diffraction peaks of Malate salt Form C

| #Peak | 2θ[°] | d-spacing [Å] | Rel. Int. [%] |
|---|---|---|---|
| 1 | 6.60 | 13.39 | 7.31 |
| 2 | 8.45 | 10.47 | 100.00 |
| 3 | 10.19 | 8.68 | 18.64 |
| 4 | 12.05 | 7.34 | 1.87 |
| 5 | 12.57 | 7.04 | 6.15 |
| 6 | 13.35 | 6.63 | 4.60 |
| 7 | 14.36 | 6.17 | 18.13 |
| 8 | 15.30 | 5.79 | 27.94 |
| 9 | 15.56 | 5.70 | 93.18 |
| 10 | 15.91 | 5.57 | 6.00 |
| 11 | 17.13 | 5.18 | 6.77 |
| 12 | 17.58 | 5.04 | 4.85 |
| 13 | 18.23 | 4.87 | 1.92 |
| 14 | 19.43 | 4.57 | 26.58 |
| 15 | 19.80 | 4.48 | 19.32 |
| 16 | 20.54 | 4.32 | 11.15 |
| 17 | 21.29 | 4.17 | 10.33 |
| 18 | 21.84 | 4.07 | 3.38 |
| 19 | 22.71 | 3.92 | 5.42 |
| 20 | 22.99 | 3.87 | 3.62 |
| 21 | 23.53 | 3.78 | 19.05 |
| 22 | 24.29 | 3.66 | 10.61 |
| 23 | 24.57 | 3.62 | 5.55 |
| 24 | 24.81 | 3.59 | 11.14 |
| 25 | 25.25 | 3.53 | 26.77 |
| 26 | 25.53 | 3.49 | 6.87 |
| 27 | 26.19 | 3.40 | 3.82 |
| 28 | 26.51 | 3.36 | 2.78 |
| 29 | 26.84 | 3.32 | 4.32 |
| 30 | 27.98 | 3.19 | 5.08 |
| 31 | 28.96 | 3.08 | 2.23 |
| 32 | 29.41 | 3.04 | 3.75 |
| 33 | 29.83 | 3.00 | 1.96 |
| 34 | 30.37 | 2.94 | 3.53 |
| 35 | 30.81 | 2.90 | 2.08 |
| 36 | 31.73 | 2.82 | 1.63 |
| 37 | 32.09 | 2.79 | 2.20 |
| 38 | 33.03 | 2.71 | 2.87 |
| 39 | 34.07 | 2.63 | 1.34 |
| 40 | 35.43 | 2.53 | 1.27 |
| 41 | 36.73 | 2.45 | 2.36 |
| 42 | 37.57 | 2.39 | 0.80 |
| 43 | 38.84 | 2.32 | 1.29 |

### Example 6—Succinate Form A

### Preparation Method:

1. Freeform Form A (560.3 mg) was weighed into a 20 mL glass vial. Ethyl acetate (14 mL) was added to dissolve Freeform Form A solids.
2. Succinic acid (8.2 mg) was weighed into an HPLC vial. Ethyl acetate solution of Freeform Form A (0.5 mL) was added into the vial.
3. The mixture was magnetically stirred at room temperature (RT) for 3 days.
4. The suspension was centrifuged (10,000 rpm, 2 min) and solids were dried at RT under vacuum for 1 day.

The resulting solid was analyzed by XRPD, TGA, and DSC as described above. The results are shown in FIGS. 5A-5C. By TGA, the sample showed weight loss of 4.92% up to 100° C. DSC showed an endotherm at 66.2° C. (peak temperature).

TABLE 9
Diffraction peaks of Succinate salt Form A

| #Peak | 2θ[°] | d-spacing [Å] | Rel. Int. [%] |
|---|---|---|---|
| 1 | 9.63 | 9.19 | 35.30 |
| 2 | 10.99 | 8.05 | 12.81 |
| 3 | 11.19 | 7.91 | 14.48 |
| 4 | 11.63 | 7.61 | 5.04 |
| 5 | 12.36 | 7.16 | 18.46 |
| 6 | 13.07 | 6.77 | 10.78 |
| 7 | 14.01 | 6.32 | 53.14 |
| 8 | 14.44 | 6.13 | 4.72 |
| 9 | 15.54 | 5.70 | 17.67 |
| 10 | 15.83 | 5.60 | 11.58 |
| 11 | 16.72 | 5.30 | 76.21 |
| 12 | 17.43 | 5.09 | 21.85 |
| 13 | 18.88 | 4.70 | 49.25 |
| 14 | 19.29 | 4.60 | 10.96 |
| 15 | 20.20 | 4.40 | 7.87 |
| 16 | 21.43 | 4.15 | 52.22 |
| 17 | 22.19 | 4.01 | 67.45 |
| 18 | 22.97 | 3.87 | 33.89 |
| 19 | 24.16 | 3.68 | 100.00 |
| 20 | 24.58 | 3.62 | 37.06 |
| 21 | 24.82 | 3.59 | 45.15 |
| 22 | 25.18 | 3.54 | 26.74 |
| 23 | 25.42 | 3.50 | 12.59 |
| 24 | 26.14 | 3.41 | 12.13 |
| 25 | 27.44 | 3.25 | 2.95 |
| 26 | 29.10 | 3.07 | 1.56 |
| 27 | 30.63 | 2.92 | 7.51 |
| 28 | 31.98 | 2.80 | 2.92 |
| 29 | 33.39 | 2.68 | 4.43 |
| 30 | 34.04 | 2.63 | 9.77 |
| 31 | 35.35 | 2.54 | 4.97 |
| 32 | 37.46 | 2.40 | 1.41 |
| 33 | 38.33 | 2.35 | 2.41 |

### Example 7—Succinate Form B

### Preparation Method:

1. Freeform Form A (560.3 mg) was weighed into a 20 mL glass vial. Methyl tert-butyl ether (14 mL) was added to dissolve Freeform Form A solids.
2. Succinic acid (8.3 mg) was weighed into an HPLC vial. Methyl tert-butyl ether solution of Freeform Form A (0.5 mL) was added into the vial.
3. The mixture was magnetically stirred at room temperature (RT) for 3 days.
4. The suspension was centrifuged (10000 rpm, 2 min) and solids were dried at RT under vacuum for 1 day.

The resulting solid was analyzed by XRPD, TGA, and DSC as described above. The results are shown in FIGS. 6A-6C. By TGA, the sample showed weight loss of 5.10% up to 100° C. DSC showed an endotherm at 90.1° C. (peak temperature).

TABLE 10
Diffraction peaks of Succinate salt Form B

| #Peak | 2θ[°] | d-spacing [Å] | Rel. Int. [%] |
|---|---|---|---|
| 1 | 10.55 | 8.39 | 52.27 |
| 2 | 11.41 | 7.75 | 15.30 |
| 3 | 12.39 | 7.14 | 9.45 |
| 4 | 14.12 | 6.27 | 8.09 |
| 5 | 15.84 | 5.59 | 100.00 |
| 6 | 16.64 | 5.33 | 16.27 |
| 7 | 17.15 | 5.17 | 6.40 |
| 8 | 17.92 | 4.95 | 7.42 |
| 9 | 18.83 | 4.71 | 7.61 |
| 10 | 19.19 | 4.63 | 22.31 |
| 11 | 19.51 | 4.55 | 6.55 |
| 12 | 20.16 | 4.40 | 14.26 |
| 13 | 21.13 | 4.20 | 20.93 |
| 14 | 22.05 | 4.03 | 7.43 |
| 15 | 23.26 | 3.82 | 13.63 |
| 16 | 24.01 | 3.71 | 13.55 |
| 17 | 24.28 | 3.67 | 21.79 |
| 18 | 24.46 | 3.64 | 19.56 |
| 19 | 25.57 | 3.48 | 4.56 |
| 20 | 26.01 | 3.43 | 6.39 |
| 21 | 26.54 | 3.36 | 7.40 |
| 22 | 27.74 | 3.22 | 4.55 |
| 23 | 28.44 | 3.14 | 4.38 |
| 24 | 30.70 | 2.91 | 2.44 |
| 25 | 32.83 | 2.73 | 1.31 |
| 26 | 38.46 | 2.34 | 1.34 |

### Example 8—Napthalene Disulfonate Form A

### Preparation Method:

1. Freeform Form A (560.1 mg) was weighed into a 20 mL glass vial. Ethanol (14 mL) was added to dissolve Freeform Form A solids.
2. Naphthalene-1,5-disulfonic acid (20.1 mg) was weighed into an HPLC vial. Ethanol solution of Freeform Form A (0.5 mL) was added into the vial.
3. The mixture was magnetically stirred at room temperature (RT) for 3 days.
4. The suspension was centrifuged (10,000 rpm, 2 min) and solids were dried at RT under vacuum for 1 day.

The resulting solid was analyzed by XRPD, TGA, and DSC as described above. The results are shown in FIGS. 7A-7C. By TGA, the sample showed weight loss of 3.61% up to 200° C. DSC showed endotherms at 94.2° C., 118.4° C., and 203.1° C. (peak temperature).

TABLE 11
Diffraction peaks of Napthalene Disulfonate salt Form A

| #Peak | 2θ[°] | d-spacing [Å] | Rel. Int. [%] |
|---|---|---|---|
| 1 | 8.38 | 10.55 | 8.61 |
| 2 | 11.96 | 7.40 | 82.87 |
| 3 | 13.00 | 6.81 | 28.61 |
| 4 | 14.06 | 6.30 | 64.91 |
| 5 | 14.94 | 5.93 | 96.07 |
| 6 | 15.42 | 5.75 | 32.46 |
| 7 | 16.79 | 5.28 | 17.33 |
| 8 | 19.13 | 4.64 | 100.00 |
| 9 | 20.60 | 4.31 | 31.26 |
| 10 | 21.10 | 4.21 | 38.51 |
| 11 | 21.44 | 4.14 | 14.77 |
| 12 | 22.41 | 3.97 | 12.20 |
| 13 | 22.79 | 3.90 | 31.62 |
| 14 | 23.85 | 3.73 | 65.37 |
| 15 | 24.00 | 3.71 | 67.02 |
| 16 | 24.35 | 3.66 | 25.21 |
| 17 | 25.53 | 3.49 | 22.12 |
| 18 | 26.15 | 3.41 | 41.80 |
| 19 | 27.25 | 3.27 | 9.95 |
| 20 | 28.01 | 3.19 | 14.35 |
| 21 | 28.42 | 3.14 | 10.71 |
| 22 | 30.32 | 2.95 | 6.87 |
| 23 | 30.97 | 2.89 | 7.79 |
| 24 | 31.84 | 2.81 | 11.56 |
| 25 | 32.46 | 2.76 | 5.47 |
| 26 | 35.68 | 2.52 | 6.14 |
| 27 | 38.98 | 2.31 | 7.06 |

### Example 9—HBr Form A

### Preparation Method:

1. Freeform Form A (560.1 mg) was weighed into a 20 mL glass vial. Acetone (14 mL) was added to dissolve Freeform Form A solids.
2. The solution (0.5 mL) was treated with hydrobromic acid (40%, 9.9 μL) at room temperature.
3. The mixture was magnetically stirred at room temperature (RT) for 2 days, then stirred at 5° C. for 1 day and −20° C. for 3 days.
4. Anti-solvent n-heptane (0.4 mL) was added into the solution. Gel-like sample was obtained.
5. The mixture was stirred with temperature cycling between 5° C. and 50° C. (3 cycles).
6. The suspension was centrifuged (10,000 rpm, 2 min) and solids were dried at RT under vacuum for 1 day.

The resulting solid was analyzed by XRPD, TGA, and DSC as described above. The results are shown in FIGS. 8A-8C. By TGA, the sample showed weight loss of 3.47% up to 150° C. DSC showed endotherms at 94.0° C. and 183.1° C. (peak temperature).

TABLE 12
Diffraction peaks of HBr salt Form A

| #Peak | 2θ[°] | d-spacing [Å] | Rel. Int. [%] |
|---|---|---|---|
| 1 | 6.28 | 14.08 | 8.55 |
| 2 | 11.95 | 7.41 | 37.40 |
| 3 | 12.56 | 7.05 | 30.69 |
| 4 | 13.64 | 6.49 | 6.28 |
| 5 | 16.23 | 5.46 | 58.51 |
| 6 | 17.03 | 5.21 | 100.00 |
| 7 | 17.47 | 5.08 | 53.94 |
| 8 | 17.78 | 4.99 | 17.37 |
| 9 | 18.87 | 4.70 | 41.63 |
| 10 | 20.83 | 4.26 | 81.90 |
| 11 | 21.20 | 4.19 | 41.85 |
| 12 | 21.65 | 4.11 | 22.50 |
| 13 | 21.94 | 4.05 | 35.66 |
| 14 | 22.67 | 3.92 | 16.70 |
| 15 | 23.13 | 3.84 | 28.65 |
| 16 | 24.01 | 3.71 | 14.61 |
| 17 | 24.32 | 3.66 | 28.38 |
| 18 | 24.86 | 3.58 | 11.86 |
| 19 | 25.19 | 3.54 | 5.91 |
| 20 | 26.15 | 3.41 | 29.26 |
| 21 | 26.65 | 3.35 | 38.22 |
| 22 | 26.93 | 3.31 | 36.43 |
| 23 | 27.16 | 3.28 | 24.23 |
| 24 | 27.39 | 3.26 | 16.01 |
| 25 | 27.65 | 3.23 | 13.56 |
| 26 | 28.05 | 3.18 | 44.56 |
| 27 | 28.46 | 3.14 | 16.34 |
| 28 | 28.84 | 3.10 | 10.88 |
| 29 | 29.21 | 3.06 | 21.76 |
| 30 | 30.03 | 2.98 | 10.83 |
| 31 | 30.94 | 2.89 | 14.35 |
| 32 | 32.20 | 2.78 | 15.29 |
| 33 | 32.70 | 2.74 | 10.20 |
| 34 | 33.46 | 2.68 | 13.47 |
| 35 | 34.04 | 2.63 | 12.20 |
| 36 | 36.20 | 2.48 | 15.09 |
| 37 | 38.78 | 2.32 | 6.29 |

### INCORPORATION BY REFERENCE

All of the U.S. patents and U.S. and PCT patent application publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are encompassed by the following claims.

1-111. (canceled)

112. A mesembrine salt, wherein the mesembrine salt is selected from the group consisting of: mesembrine malate, mesembrine naphthalene disulfonate, mesembrine HBr, mesembrine tartrate, mesembrine fumarate, and mesembrine succinate.

113. The mesembrine salt of claim 112, wherein the mesembrine salt is a mesembrine malate salt having an X-ray powder diffraction (XRPD) pattern comprising the following 2-theta peaks (2 theta±0.2): 8.6°, 16.0°, 19.6°, 19.7°, and 25.3°.

114. The mesembrine salt of claim 112, wherein the mesembrine salt is a (−) mesembrine malate salt, characterized by one or more of the following:
a. an X-ray powder diffraction (XRPD) having the following 2-theta peaks (2 theta±0.2) and corresponding d-spacing (angstroms±0.2):

| 2θ ± 0.2 [°] | d-spacing ± 0.2 [Å] |
|---|---|
| 8.6 | 10.3 |
| 16.0 | 5.6 |
| 19.6 | 4.5 |
| 19.7 | 4.5 |
| 25.3 | 3.5; |

b. a sample weight loss of up to about 8.4% upon heating up to 100° C. measured by TGA according to the following parameters:

| | |
|---|---|
| Parameters | TGA |
| Method | Ramp |
| Sample pan | Aluminum, open |
| Temperature | RT-350° C. |
| Heating rate | 10° C./min |
| Purge gas | N₂ |

and
c. a DSC having an endotherm at about 70° C. to about 75° C. (peak temperature), measured by DSC according to the following parameters:

| | |
|---|---|
| Parameters | DSC |
| Method | Ramp |
| Sample pan | Aluminum, crimped |
| Temperature | 25° C.-230° C. |
| Heating rate | 10° C./min |
| Purge gas | N₂. |

115. The mesembrine salt of claim 112, wherein the mesembrine salt is a mesembrine malate salt, and the mesembrine malate salt is solid Form A.

116. The mesembrine salt of claim 112, wherein the mesembrine salt is a mesembrine malate salt having an X-ray powder diffraction (XRPD) pattern comprising the following 2-theta peaks (2 theta±0.2): 8.4°, 15.3°, 15.6°, 19.4°, and 25.2°.

117. The mesembrine salt of claim 112, wherein the mesembrine salt is a (−) mesembrine malate salt, characterized by one or more of the following:
a. an X-ray powder diffraction (XRPD) having the following 2-theta peaks (2 theta±0.2) and corresponding d-spacing (angstroms±0.2):

| 2θ ± 0.2 [°] | d-spacing ± 0.2 [Å] |
|---|---|
| 8.4 | 10.5 |
| 15.3 | 5.8 |
| 15.6 | 5.7 |
| 19.4 | 4.6 |
| 25.2 | 3.5 |

b. a sample weight loss of up to about 8.4% upon heating up to 100° C. measured by TGA according to the following parameters:

| | |
|---|---|
| Parameters | TGA |
| Method | Ramp |
| Sample pan | Aluminum, open |
| Temperature | RT-350° C. |
| Heating rate | 10° C./min |
| Purge gas | N₂ |

and
c. a DSC having an endotherm at about 70° C. to about 75° C. (peak temperature), measured by DSC according to the following parameters:

| | |
|---|---|
| Parameters | DSC |
| Method | Ramp |
| Sample pan | Aluminum, crimped |
| Temperature | 25° C.-230° C. |
| Heating rate | 10° C./min |
| Purge gas | N₂. |

118. The mesembrine salt of claim 112, wherein the mesembrine salt is a mesembrine malate salt, and the mesembrine malate salt is solid Form C.

119. The mesembrine salt of claim 112, wherein the mesembrine salt is mesembrine naphthalene disulfonate.

120. The mesembrine salt of claim 112, wherein the mesembrine salt is a mesembrine naphthalene disulfonate salt having an X-ray powder diffraction (XRPD) pattern comprising the following 2-theta peaks (2 theta±0.2): 12.0°, 14.9°, 19.1°, 23.8°, and 24.0°.

121. The mesembrine salt of claim 112, wherein the mesembrine salt is a (−) mesembrine naphthalene disulfonate salt, characterized by one or more of the following:
a. an X-ray powder diffraction (XRPD) having the following 2-theta peaks (2 theta±0.2) and corresponding d-spacing (angstroms±0.2):

| 2θ ± 0.2 [°] | d-spacing ± 0.2 [Å] |
|---|---|
| 12.0 | 7.4 |
| 14.9 | 5.9 |
| 19.1 | 4.6 |
| 23.8 | 3.7 |
| 24.0 | 3.7 |

b. a sample weight loss of up to about 3.6% upon heating up to 200° C. measured by TGA according to the following parameters:

| | |
|---|---|
| Parameters | TGA |
| Method | Ramp |
| Sample pan | Aluminum, open |
| Temperature | RT-350° C. |
| Heating rate | 10° C./min |
| Purge gas | N₂ |

and
c. a DSC having an endotherm at about 92° C. to about 97° C., and/or at about 116° C. to about 121° C., and/or at about 200° C. to about 205° C., measured by DSC according to the following parameters:

| | |
|---|---|
| Parameters | DSC |
| Method | Ramp |
| Sample pan | Aluminum, crimped |
| Temperature | 25° C.-230° C. |
| Heating rate | 10° C./min |
| Purge gas | N₂ |

122. The mesembrine salt of claim 112, wherein the mesembrine salt is a mesembrine naphthalene disulfonate salt, and the mesembrine naphthalene disulfonate salt is solid Form A.

123. The mesembrine salt of claim 112, wherein the mesembrine salt is a mesembrine HBr salt having an X-ray powder diffraction (XRPD) pattern comprising the following 2-theta peaks (2 theta±0.2): 16.2°, 17.0°, 17.5°, 21.2°, and 28.0°.

124. The mesembrine salt of claim 112, wherein the mesembrine salt is a (−) mesembrine HBr salt, characterized by one or more of the following:
a. an X-ray powder diffraction (XRPD) having the following 2-theta peaks (2 theta±0.2) and corresponding d-spacing (angstroms±0.2):

| 2θ ± 0.2 [°] | d-spacing ± 0.2 [Å] |
|---|---|
| 16.2 | 5.5 |
| 17.0 | 5.2 |
| 17.5 | 5.1 |
| 21.2 | 4.2 |
| 28.0 | 3.2 |

b. a sample weight loss of up to about 3.5% upon heating up to 150° C. measured by TGA according to the following parameters:

| | |
|---|---|
| Parameters | TGA |
| Method | Ramp |
| Sample pan | Aluminum, open |
| Temperature | RT-350° C. |
| Heating rate | 10° C./min |
| Purge gas | N₂ |

and
c. a DSC having an endotherm at about 92° C. to about 97° C., and/or at about 180° C. to about 185° C.° C. (peak temperature), measured by DSC according to the following parameters:

| | |
|---|---|
| Parameters | DSC |
| Method | Ramp |
| Sample pan | Aluminum, crimped |
| Temperature | 25° C.-230° C. |
| Heating rate | 10° C./min |
| Purge gas | N₂ |

125. The mesembrine salt of claim 112, wherein the mesembrine salt is a mesembrine HBr salt, and the mesembrine HBr salt is solid Form A.

126. The mesembrine salt of claim 112, wherein the mesembrine salt is selected from the group consisting of:
a. a mesembrine tartrate salt having an X-ray powder diffraction (XRPD) pattern comprising the following 2-theta peaks (2 theta±0.2): 10.5°, 15.2°, 16.7°, 18.3°, and 21.0°;
b. a mesembrine fumarate salt having an X-ray powder diffraction (XRPD) pattern comprising the following 2-theta peaks (2 theta±0.2): 9.1°, 17.0°, 17.3°, 23.7°, and 24.2°;
c. a mesembrine succinate salt having an X-ray powder diffraction (XRPD) pattern comprising the following 2-theta peaks (2 theta±0.2): 14.0°, 16.7°, 21.4°, 22.2°, and 24.2°; and
d. a mesembrine succinate salt having an X-ray powder diffraction (XRPD) pattern comprising the following 2-theta peaks (2 theta±0.2): 10.6°, 15.8°, 19.2°, 21.1°, and 24.3°.

127. The mesembrine salt of claim 126, wherein the mesembrine salt is a mesembrine tartrate salt, and the mesembrine tartrate salt is Form B.

128. The mesembrine salt of claim 126, wherein the mesembrine salt is a mesembrine fumarate salt, and the mesembrine fumarate salt is Form A.

129. The mesembrine salt of claim 126, wherein the mesembrine salt is a mesembrine succinate salt, and the mesembrine succinate salt is Form A or Form B.

130. A pharmaceutical composition, comprising a mesembrine salt of claim 112, and a pharmaceutically acceptable excipient.

131. A method of treating social anxiety disorder, generalized anxiety disorder or depression, the method comprising administering to a mammal in need thereof an effective amount of a mesembrine salt according to claim 112.
